(19) United States
(12) Patent Application Publication
(10) Pub. No.: US 2024/0115585 A1
(43) Pub. Date: Apr. 11, 2024

(54) METHODS OF TREATING CANCER

(71) Applicant: NeuPharma, Inc., San Mateo, CA (US)

(72) Inventor: Xiangping QIAN, Foster City, CA (US)

(21) Appl. No.: 18/524,489

(22) Filed: Nov. 30, 2023

Related U.S. Application Data
(63) Continuation of application No. 16/772,755, filed on Jun. 12, 2020.
(60) Provisional application No. 62/599,643, filed on Dec. 15, 2017.

(51) Int. Cl. A61K 31/585

(52) U.S. Cl. CPC A61K 31/585; A61K 9/0019

(57) ABSTRACT

The present invention relates to methods of treating cancer comprising administering a bufalin derivative compound of Formula I, wherein the compound is administered at least once a week for at least two weeks

### CROSS-REFERENCE TO RELATED APPLICATIONS

This application claims priority benefit of U.S. Provisional Patent Application No. 62/599.643, filed Dec. 15, 2017. The entire contents of that application are hereby incorporated by reference herein.

### FIELD OF THE INVENTION

This invention pertains to methods and compositions for the treatment of cancer by administering a bufalin derivative compound.

### BACKGROUND OF THE INVENTION

Despite significant advances in early detection and multi-modal therapies for the treatment of cancer, an unmet medical need exists for the treatment of patients with advanced cancers.

The traditional Chinese medicine Huachansu (an injectable form of chansu) is used in China for the treatment of a variety of cancers including liver, lung, pancreatic and colorectal cancer. Its anti-cancer activity is attributed to three major steroidal cardiac glycosides: bufalin, resibufogenin and cinobufagin. When tested, bufalin demonstrated strong activity in cancer cell lines and animal models. WO2011/085641 discloses various bufalin derivatives which were shown to be particularly effective in cancer treatment. These compounds find tremendous potential in providing effective therapeutic options for cancer patients.

The disclosures of all publications, patents, patent applications, and published patent applications referred to herein are hereby incorporated herein by reference in their entirety.

### BRIEF SUMMARY OF THE INVENTION

The present invention provides a method of treating cancer in an individual, comprising administering to the individual an effective amount of a compound of Formula I:

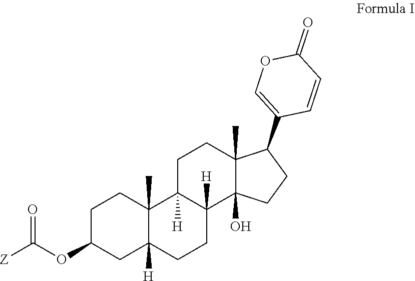

or a pharmaceutically acceptable salt thereof, wherein: Z is —OR¹ or —NR²R³; R¹ is optionally substituted alkyl, optionally substituted cycloalkyl, optionally substituted heterocycloalkyl, optionally substituted aryl, or optionally substituted heteroaryl; R¹ is hydrogen, optionally substituted alkyl, optionally substituted cycloalkyl, optionally substituted heterocycloalkyl, optionally substituted aryl, or optionally substituted heteroaryl; and R³ is optionally substituted alkyl, optionally substituted cycloalkyl, optionally substituted heterocycloalkyl, optionally substituted aryl, or optionally substituted heteroaryl, or R² and R³ are taken together with the atom to which they are attached to form an optionally substituted heterocycloalkyl; and the compound is administered at least once a week for at least two weeks.

In some embodiments, the compound is administered at least two times a week, including for example, at least three times a week. In some embodiments, the compound is administered three times a week.

In some embodiments according to any aforementioned method, the compound is administered for at least three consecutive days during a week, including for example, on days 1, 2, and 3 of each week. In some embodiments, the compound is administered once every other day during a week, including for example, on days 1, 3, and 5 of each week. In some embodiments, the compound is administered daily for at least two weeks.

In some embodiments, the compound is administered at least once a week for at least two weeks, including for example, at least once a week for two weeks with one week of rest in a 21-day dosage cycle.

In some embodiments, the compound is administered at least once a week for at least three weeks, including for example, at least once a week for three weeks with one week of rest in a 28-day dosage cycle.

In some embodiments according to any aforementioned method, the compound is administered at the dosage range of about 0.01 mg/m² to about 2 mg/m².

In some embodiments according to any aforementioned method, the compound is administered at the dosage range of about 0.2 mg/m² to about 2 mg/m².

In some embodiments according to any aforementioned method, the total dose of the compound administered to the individual is at least about 0.4 mg/m² per week.

In some embodiments according to any aforementioned method, the cancer is selected from the group consisting of colorectal cancer, liver cancer, lung cancer, breast cancer, prostate cancer, pancreatic cancer, and oral cancer. In some embodiments, the cancer is metastatic cancer.

In some embodiments according to any aforementioned method, the compound is administered intravenously, including for example, infusion for at least two hours at each administration or continuously by infusion for at least 24 hours at each administration

In some embodiments according to any aforementioned method, the individual expresses (e.g., highly expresses) PI3K and/or pAKT.

In some embodiments according to any aforementioned method, the individual has progressed on or failed at least one prior therapy (such as systemic therapy).

In some embodiments according to any aforementioned method, the compound is administered at the concentration of about 0.002 mg/mL to about 0.01 mg/mL.

In some embodiments according to any aforementioned method, the compound is in a pharmaceutical composition comprising the compound and a pharmaceutically acceptable carrier. In some embodiments, the pharmaceutically acceptable carrier comprises an acetate or acetic acid.

In some embodiments according to any aforementioned method, the individual is human.

In some embodiments according to any aforementioned method, the compound is of Formula I, or a pharmaceutically acceptable salt thereof, wherein Z is —OR¹ or —NR²R³. In some embodiments, Z is —OR¹, wherein R¹ is optionally substituted alkyl, optionally substituted cycloalkyl, or optionally substituted heterocycloalkyl. In some embodiments, Z is —NR²R³, wherein R¹ is hydrogen, optionally substituted alkyl, optionally substituted cycloalkyl, or optionally substituted heterocycloalkyl, and R³ is optionally substituted alkyl, optionally substituted cycloalkyl, or optionally substituted heterocycloalkyl or R² and R³ are taken together with the atom to which they are attached to form a 4- to 8-membered heterocycloalkyl. In one aspect, R² is hydrogen and R¹ is optionally substituted alkyl.

In some embodiments according to any aforementioned method, the compound is selected from Compound Nos. 1-7, or a pharmaceutically acceptable salt thereof:

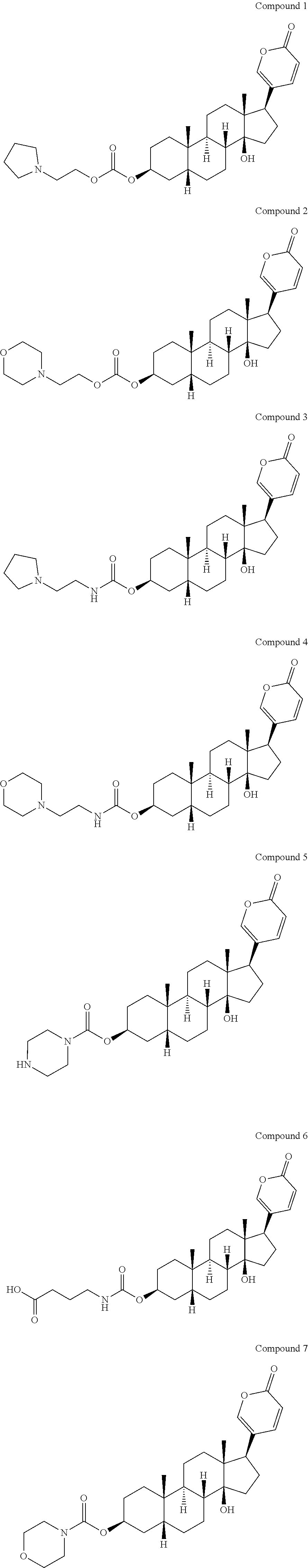

The methods described herein can be used for any one or more of the following purposes: alleviating one or more symptoms of a cancer, delaying progressing of a cancer, shrinking tumor size in a cancer patient, inhibiting cancer growth, prolonging overall survival, prolonging disease-free survival, prolonging time to tumor progression, preventing or delaying metastasis, reducing (such as eradicating) preexisting metastasis, reducing incidence or burden of preexisting metastasis, and preventing recurrence of cancer.

### DETAILED DESCRIPTION OF THE INVENTION

The present invention provides methods of cancer treatment based on specific dosing regimens which are particularly effective in treating cancer. The methods comprise administration of a compound of Formula I:

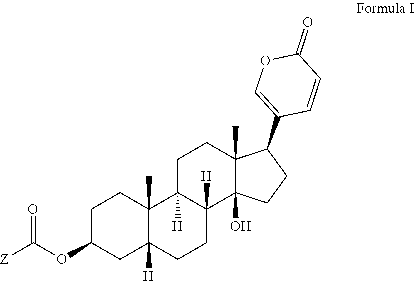

or a pharmaceutically acceptable salt thereof, wherein:
Z is —OR¹ or —NR²R³;

R¹ is optionally substituted alkyl, optionally substituted cycloalkyl, optionally substituted heterocycloalkyl, optionally substituted aryl, or optionally substituted heteroaryl; R² is hydrogen, optionally substituted alkyl, optionally substituted cycloalkyl, optionally substituted heterocycloalkyl, optionally substituted aryl, or optionally substituted heteroaryl; and R³ is optionally substituted alkyl, optionally substituted cycloalkyl, optionally substituted heterocycloalkyl, optionally substituted aryl, or optionally substituted heteroaryl, or R² and R³ are taken together with the nitrogen atom to which they are attached to form an optionally substituted heterocycloalkyl.

The dosing regimens in some embodiments comprise administering the compound of Formula I or pharmaceutically acceptable salt thereof at least once a week for at least two weeks or at least three weeks. In some embodiments, the compound of Formula I or pharmaceutically acceptable salt thereof is administered for at least three days during a week (for example for at least two weeks or at least three weeks) Without being bound by theory, it is believed that Increase in dosing frequency (for example by administering the compound daily for at least three days during a week) would be very effective in cancer treatment. The methods are particularly suitable for treating cancer, such as solid tumor, including for example liver cancer, colorectal cancer, and lung cancer.

The present invention thus provides various methods of cancer treatment by following the inventive dosing regimens. Also provided are compositions (such as pharmaceutical compositions), kits, and unit dosages useful for the methods described herein.

### Definitions

As used herein, when any variable occurs more than one time in a chemical formula, its definition on each occurrence is independent of its definition at every other occurrence.

As used herein, a dash (“-”) that is not between two letters or symbols is used to indicate a point of attachment for a substituent. For example, —CONH₂ is attached through the carbon atom.

As used herein, “optional” or “optionally” is meant that the subsequently described event or circumstance may or may not occur, and that the description includes instances wherein the event or circumstance occurs and instances in which it does not. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, “alkyl” refers to straight chain and branched chain having the indicated number of carbon atoms, usually from 1 to 20 carbon atoms, for example 1 to 8 carbon atoms, such as 1 to 6 carbon atoms For example C₁-C₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “butyl” is meant to include n-butyl, sec-butyl, isobutyl and t-butyl; “propyl” includes n-propyl and isopropyl. “Lower alkyl” refers to alkyl groups having one to six carbons. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is a subset of alkyl, referring to the same residues as alkyl, but having two points of attachment. Alkylene groups will usually have from 2 to 20 carbon atoms, for example 2 to 8 carbon atoms, such as from 2 to 6 carbon atoms. For example, C₀ alkylene indicates a covalent bond and C₁ alkylene is a methylene group.

As used herein, “alkeny!” refers to an unsaturated branched or straight-chain alkyl group having at least one carbon-carbon double bond derived by the removal of one molecule of hydrogen from adjacent carbon atoms of the parent alkyl. The group may be in either the cis or trans configuration about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl; and the like. In certain embodiments, an alkenyl group bas from 2 to 20 carbon atoms and in other embodiments, from 2 to 6 carbon atoms. “Lower alkenyl” refers to alkenyl groups having two to six carbons.

As used herein, “alkynyl” refers to an unsaturated branched or straight-chain alkyl group having at least one carbon-carbon triple bond derived by the removal of two molecules of hydrogen from adjacent carbon atoms of the parent alkyl. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group bas from 2 to 20 carbon atoms and in other embodiments, from 3 to 6 carbon atoms. “Lower alkynyl” refers to alkynyl groups having two to six carbons.

As used herein, “cycloalkyl” refers to a non-aromatic carbocyclic ring, usually having from 3 to 7 ring carbon atoms. The ring may be saturated or have one or more carbon-carbon double bonds. Examples of cycloalkyl groups include cyclopropyl, cyclobutyl, cyclopentyl, cyclopentenyl, cyclohexyl, and cyclohexenyl, as well as bridged and caged ring groups such as norbornane.

As used herein, “alkoxy” refers to an alkyl group of the indicated number of carbon atoms attached through an oxygen bridge such as, for example, methoxy, ethoxy, propoxy, isopropoxy, n-butoxy, sec-butoxy, tert-butoxy, pentyloxy, 2-pentyloxy, isopentyloxy, neopentyloxy, hexyloxy, 2-hexyloxy, 3-hexyloxy, 3-methylpentyloxy, and the like. Alkoxy groups will usually have from 1 to 7 carbon atoms attached through the oxygen bridge. “Lower alkoxy” refers to alkoxy groups having one to six carbons.

As used herein, “acyl” refers to the groups H—C(O)—; (alkyl)-C(O)—; (cycloalkyl)-C(O)—; (aryl)-C(O)—; (heteroaryl)-C(O)—; and (heterocycloalkyl)-C(O)—, wherein the group is attached to the parent structure through the carbonyl functionality and wherein alkyl, cycloalkyl, aryl, heteroaryl, and heterocycloalkyl are as described herein. Acyl groups have the indicated number of carbon atoms, with the carbon of the keto group being included in the numbered carbon atoms. For example a C₂ acyl group is an acetyl group having the formula CH₃(C═O)—.

As used herein, “formyl” refers to the group —C(O)H.

As used herein, “alkoxycarbonyl” refers to a group of the formula (alkoxy)(C═O))— attached through the carbonyl carbon wherein the alkoxy group has the indicated number of carbon atoms. Thus a C₁-C₆ alkoxy carbonyl group is an alkoxy group having from 1 to 6 carbon atoms attached through its oxygen to a carbonyl linker.

As used herein, “azido” refers to the group —N₃.

As used herein, “amino” refers to the group —NH₂.

As used herein, “mono- and di-(alkyl)amino” refers to secondary and tertiary alkyl amino groups, wherein the alkyl groups are as defined above and have the indicated number of carbon atoms. The point of attachment of the alkylamino group is on the nitrogen. Examples of mono- and di-alkylamino groups include ethylamino, dimethylamino, and methyl-propyl-amino.

As used herein, “aminocarbonyl” refers to the group —CONR^{b}R^{c}, where
R^{b} is H, optionally substituted C₁-C₆ alkyl, optionally substituted cycloalkyl, optionally substituted heterocycloalkyl, optionally substituted aryl, optionally substituted heteroaryl, of optionally substituted alkoxy; and
R^{c} is hydrogen or optionally substituted C₁-C₄ alkyl; or
R^{b} and R^{c} taken together with the nitrogen to which they are bound, form an optionally substituted 4- to 8-membered nitrogen-containing heterocycloalkyl which optionally includes 1 or 2 additional heteroatoms chosen from O, N, and S in the heterocycloalkyl ring;
where each substituted group is independently substituted with one or more substituents independently C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, −SO₂(C₁-C₄ alkyl), −SO₂(phenyl), —SO₂(C₁-C₄ haloalkyl), —SO₂NH, —SO₂NH(C₁-C₄ alkyl), —SO₂NH(phenyl), —NHSO₂(C₁-C₄ alkyl), —NHSO₂(phenyl), or —NHSO₂(C₁-C₄ haloalkyl).

As used herein, “aryl” refers to: 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene.

For example, aryl includes 6-membered carbocyclic aromatic rings fused to a 4- to 8-membered heterocycloalkyl ring containing I or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent poly cyclic hydrocarbon radicals whose names end in “-yl” by removal of one hydrogen atom from the carbon atom with the free valence are named by adding “-idene” to the name of the corresponding univalent radical, e.g. a naphthyl group with two points of attachment is termed naphthylidene. Aryl, however, does not encompass or overlap in any way with heteroaryl, separately defined below. Hence, if one or more carbocyclic aromatic rings is fused with a heteroaryl ring, the resulting ring system is heteroaryl, not aryl, as defined herein.

As used herein, “aryloxy” refers to the group —O-aryl

As used herein, “aralkyl” refers to the group -alkyl-aryl.

As used herein, “carbamimidoyl” refers to the group —C(═NH)—NH₂.

As used herein, “substituted carbamimidoyl” refers to the group —C(═NR^{c})—NR^{f}R^{g} where
R^{c} is hydrogen, cyano, optionally substituted alkyl, optionally substituted cycloalkyl, optionally substituted aryl, optionally substituted heteroaryl, or optionally substituted heterocycloalkyl; and
R^{f} and R^{g} are independently hydrogen optionally substituted alkyl, optionally substituted cycloalkyl, optionally substituted aryl, optionally substituted heteroaryl, or optionally substituted heterocycloalkyl,
provided that at least one of R^{c}, R^{f} , and R^{g} is not hydrogen and wherein substituted alkyl, cycloalkyl, aryl, heterocycloalkyl, and heteroaryl refer respectively to alkyl, cycloalkyl, aryl, heterocycloalkyl, and heteroaryl wherein one or more (such as up to 5, for example, up to 3) hydrogen atoms are replaced by a substituent independently —R^{c}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substituent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OP(O)(OR^{b})OR^{c}, sulfanyl (such as SR^{b}), sulfinyl (such as —SOR^{a}), or sulfonyl (such as —SO₂R^{a} and —SO₂NR^{b}R^{c}),
where R^{a} is optionally substituted C₁-C₆ alkyl, optionally substituted aryl, or optionally substituted heteroaryl;
R^{b} is H, optionally substituted C₁-C₆ alkyl, optionally substituted aryl, or optionally substituted heteroaryl; and
R^{c} is hydrogen or optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), NH(C₁-C₄ alkylphenyl), cyano, nitro, oxo (as a substituent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), or —NHSO₂(C₁-C₄ haloalkyl).

As used herein, “halo” refers to fluoro, chloro, bromo, and iodo, and the term “halogen” includes fluorine, chlorine, bromine, and iodine.

As used herein, “haloalkyl” refers to alkyl as defined above having the specified number of carbon atoms, substituted with 1 or more halogen atoms, up to the maximum allowable number of halogen atoms. Examples of haloalkyl include, but are not limited to, trifluoromethyl, difluoromethyl, 2-fluoroethyl, and penta-fluoroethyl.

As used herein, “heteroaryl” refers to:
5- to 7-membered aromatic, monocyclic rings containing one or more, for example, from 1 to 4, or in certain embodiments, from 1 to 3, heteroatoms chosen from N, O, and S, with the remaining ring atoms being carbon;
bicyclic rings containing one or more, for example, from 1 to 4, or in certain embodiments, from 1 to 3, heteroatoms chosen from N, O, and S, with the remaining ring atoms being carbon and wherein at least one heteroatom is present in an aromatic ring, and
tricyclic rings containing one or more, for example, from 1 to 5, or in certain embodiments, from 1 to 4, heteroatoms chosen from N, O, and S, with the remaining ring atoms being carbon and wherein at least one heteroatom is present in an aromatic ring.

For example, heteroaryl includes a 5- to 7-membered heterocycloalkyl, aromatic ring fused to a 4- to 8-membered cycloalkyl or heterocycloalkyl ring. For such fused, bicyclic heteroaryl ring systems wherein only one of the rings contains one or more heteroatoms, the point of attachment may be at either ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Examples of heteroaryl groups include, but are not limited to, pyridyl, pyrazinyl, pyrimidinyl, pyrazolinyl, imidazolyl, isoxazolyl, oxazolyl, thiazolyl, thiadiazolyl, tetrazolyl, thienyl, benzothiophenyl, furanyl, pyrrolyl, benzofuranyl, benzoimidazolyl, indolyl, pyridazinyl, triazolyl, quinolinyl, quinoxalinyl, pyrazolyl, and 5,6,7,8-tetrahydroisoquinolinyl. Bivalent radicals derived from univalent heteroaryl radicals whose names end in “-yl” by removal of one hydrogen atom from the atom with the free valence are named by adding “-idene” to the name of the corresponding univalent radical, e.g. a pyridyl group with two points of attachment is a pyridylidene. Heteroaryl does not encompass or overlap with aryl, cycloalkyl, or heterocycloalkyl, as defined herein.

Substituted heteroaryl also includes ring systems substituted with one or more oxide (—O⁻) substituents, such as pyridinyl N-oxides.

As used herein, “heterocycloalkyl” refers to a single, non-aromatic ring, usually with 3 to 8 ring atoms, containing at least 2 carbon atoms in addition to 1-3 heteroatoms independently chosen from oxygen, sulfur, and nitrogen, as well as combinations comprising at least one of the foregoing heteroatoms. The ring may be saturated or have one or more carbon-carbon double bonds. Suitable heterocycloalkyl groups include but are not limited to, for example, pyrrolidinyl, morpholinyl, piperidinyl, piperazinyl, azetidinyl, diazepanyl, diazocanyl, pyrrolidinyl, morpholinyl, piperidinyl, piperazinyl, imidazolidinyl, pyrazolidinyl, dihydrofuranyl, and tetrahydrofuranyl. Substituted heterocycloalkyl can also include ring systems substituted with one or more oxo (═O) or oxide (—O⁻) substituents, such as piperidinyl N-oxide, morpholinyl-N-oxide, 1-oxo-1-thiomorpholinyl and 1,1-dioxo-1-thiomorpholinyl.

“Heterocycloalkyl” also includes bicyclic ring systems wherein one non-aromatic ring, usually with 3 to 7 ring atoms, contains at least 2 carbon atoms in addition to 1-3 heteroatoms independently chosen from oxygen, sulfur, and nitrogen, as well as combinations comprising at least one of the foregoing heteroatoms; and the other ring, usually with 3 to 7 ring atoms, optionally contains 1-3 heteratoms independently chosen from oxygen, sulfur, and nitrogen and is not aromatic.

As used herein, “sulfanyl” refers to the groups: —S-(optionally substituted (C₁-C₆)alkyl), —S-(optionally substituted cycloalkyl), —S-(optionally substituted aryl), —S-(optionally substituted heteroaryl), and —S-(optionally substituted heterocycloalkyl). Hence, sulfanyl includes the group C₁-C₆ alkylsulfanyl.

As used herein, “sulfinyl” refers to the groups: —S(O)-(optionally substituted (C₁-C₆)alkyl), —S(O)-(optionally substituted cycloalkyl), —S(O)-(optionally substituted aryl), —S(O)-optionally substituted heteroaryl), —S(O)-(optionally substituted heterocycloalkyl); and —S(O)-(optionally substituted amino).

As used herein, “sulfonyl” refers to the groups: —S(O₂)-(optionally substituted (C₁-C₆)alkyl), —S(O₂)-(optionally substituted cycloalkyl), —S(O₂)-(optionally substituted aryl), —S(O₂)-(optionally substituted heteroaryl), —S(O₂)-(optionally substituted heterocycloalkyl), and —S(O₂)-(optionally substituted amino).

As used herein, “substituted” refers to any one or more hydrogens on the designated atom or group is replaced with a selection from the indicated group, provided that the designated atom's normal valence is not exceeded. When a substituent is oxo (i.e. ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation as an agent having at least practical utility. Unless otherwise specified, substituents are named into the core structure. For example, it is to be understood that when (cycloalkyl)alkyl is listed as a possible substituent, the point of attachment of this substituent to the core structure is in the alky 1 portion.

As used herein, the terms “substituted” alkyl, cycloalkyl, aryl, heterocycloalkyl, and heteroaryl, unless otherwise expressly defined, refer respectively to alkyl, cycloalkyl, aryl, heterocycloalkyl, and heteroaryl wherein one or more (such as up to 5, for example, up to 3) hydrogen atoms are replaced by a substituent independently —R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, azido, nitro, oxo (as a substituent for cycloalkyl or heterocycloalkyl), optionally substituted acyl (such as —CO₂R^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OP(^{O})(O^{Rb})OR^{c} sulfanyl (such as SR^{b}), sulfinyl (such as —SOR^{a}) or sulfonyl (such as —SO₂R^{a} and —SO₂NR^{b}R^{c}),
where R^{a} is optionally substituted C₁-C₆ alkyl, optionally substituted cycloalkyl, optionally substituted heterocycloalkyl, optionally substituted alkenyl, optionally substituted alkynyl, optionally substituted aryl, or optionally substituted heteroaryl; R^{b} is hydrogen, optionally substituted C₁-C₆ alkyl, optionally substituted cycloalkyl, optionally substituted heterocycloalkyl, optionally substituted aryl, or optionally substituted heteroaryl; and
R^{c} is hydrogen or optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl or heterocycloalkyl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), or —NHSO₂(C₁-C₄ haloalkyl).

As used herein, “substituted acyl” refers to the groups (substituted alkyl)-C(O)—; (substituted cycloalkyl)-C(O)—; (substituted aryl)-C(O)—; (substituted heteroaryl)-C(O)—; and (substituted heterocycloalkyl)-C(O)—, wherein the group is attached to the parent structure through the carbonyl functionality and wherein substituted alkyl, cycloalkyl, aryl, heteroaryl, and heterocycloalkyl, refer respectively to alkyl, cycloalkyl, aryl, heteroaryl, and heterocycloalkyl wherein one or more (such as up to 5, for example, up to 3) hydrogen atoms are replaced by a substituent independently —R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substituent for cycloalkyl or heterocycloalkyl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OP(O)(OR^{b})OR^{c}, sulfanyl (such as SR^{b}), sulfinyl (such as —SOR^{a}), or sulfonyl (such as —SO₂R^{a} and —SO₂NR^{b}R^{c}),
where R^{a} is optionally substituted C₁-C₄ alkyl, optionally substituted alkenyl, optionally substituted alkynyl, optionally substituted aryl, or optionally substituted heteroaryl;
R^{b} is H, optionally substituted C₁-C₄ alkyl, optionally substituted cycloalkyl, optionally substituted heterocycloalkyl, optionally substituted aryl, or optionally substituted heteroaryl; and
R^{c} is hydrogen or optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl or heterocycloalkyl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), or —NHSO₂(C₁-C₄ haloalkyl).

As used herein, “substituted alkoxy” refers to alkoxy wherein the alkyl constituent is substituted (i.e. —O-(substituted alkyl)) wherein “substituted alkyl” refers to alkyl wherein one or more (such as up to 5, for example, up to 3) hydrogen atoms are replaced by a substituent independently chosen from —R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substituent for cycloalkyl or heterocycloalkyl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OP(O)(OR^{b})OR^{c}, sulfanyl (such as SR^{b}), sulfinyl (such as —SOR^{a}), and sulfonyl (such as —SO₂R^{a} and —SO₂NR^{b}R^{c}),
where R^{a} is optionally substituted C₁-C₆ alkyl, optionally substituted alkenyl, optionally substituted alkynyl, optionally substituted aryl, or optionally substituted heteroaryl;
R^{b} is H, optionally substituted C₁-C₆ alkyl, optionally substituted cycloalkyl, optionally substituted heterocycloalkyl, optionally substituted aryl, or optionally substituted heteroaryl; and
R^{c} is hydrogen or optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ , alkyl), —N(C₁-C₄ alkyl)(C₁-C₄ alkylphenyl), —NH(C₁-C₄ alkylphenyl), cyano, nitro, oxo (as a substituent for cycloalkyl or heterocycloalkyl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), or —NHSO₂(C₁-C₄ haloalkyl).

In some embodiments, a substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and O(CH₂CH₂O)ₓCH₃, where x is an integer of 2-20, such as 2-10, and for example, 2-5. Another substituted alkoxy group is hydroxyalkoxy or —OCH₂(CH₂)_{y}OH, where y is an integer of 1-10, such as 1-4.

As used herein, “substituted alkoxycarbonyl” refers to the group (substituted alkyl)-O—C(O)— wherein the group is attached to the parent structure through the carbonyl functionality and wherein substituted refers to alkyl wherein one or more (such as up to 5. for example, up to 3) hydrogen atoms are replaced by a substituent independently —R^{a}, —OR^{b}, optionally substituted amino (including —NR^{c}COR^{b}, —NR^{c}CO₂R^{c}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substituent for cycloalkyl or heterocycloalkyl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OP(O)(OR^{b})OR^{c}, sulfanyl (such as SR^{b}), sulfinyl (such as —SOR^{a}), and sulfonyl (such as —SO₂R^{a} and —SO₂NR^{b}R^{c}),
where R^{a} is optionally substituted C₁-C₆ alkyl, optionally substituted alkenyl, optionally substituted alkynyl, optionally substituted aryl, or optionally substituted heteroaryl;
R^{b} is H, optionally substituted C₁-C₆ alkyl, optionally substituted cycloalkyl, optionally substituted heterocycloalkyl, optionally substituted aryl, or optionally substituted heteroaryl; and
R^{c} is hydrogen or optionally substituted C₁-C₄ alkyl; or
R^{b} and R^{c}, and the nitrogen to which they are attached, form an optionally substituted heterocycloalkyl group; and
where each optionally substituted group is unsubstituted or independently substituted with one or more, such as one, two, or three, substituents independently C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl or heterocycloalkyl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), or —NHSO₂(C₁-C₄ haloalkyl).

As used herein, “substituted amino” refers to the group —NHR^{d} or —NR^{d}R^{e} wherein R^{d} is hydroxyl, formyl, optionally substituted alkoxy, optionally substituted alkyl, optionally substituted cycloalkyl, optionally substituted acyl, optionally substituted carbamimidoyl, aminocarbonyl, optionally substituted aryl, optionally substituted heteroaryl, optionally substituted heterocycloalkyl, optionally substituted alkoxycarbonyl, sulfinyl and sulfonyl, and wherein R^{e} is chosen from optionally substituted alkyl, optionally substituted cycloalkyl, optionally substituted aryl, optionally substituted heteroaryl, or optionally substituted heterocycloalkyl, and wherein substituted alkyl, cycloalkyl, aryl, heterocycloalkyl, and heteroaryl refer respectively to alkyl, cycloalkyl, aryl, heterocycloalkyl, and heteroaryl wherein one or more (such as up to 5, for example, up to 3) hydrogen atoms are replaced by a substituent independently —R^{a}, 13 OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substituent for cycloalkyl or heterocycloalkyl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OP(O)(OR^{b})OR^{c}, sulfanyl (such as SR^{b}), sulfinyl (such as —SOR^{a}), or sulfonyl (such as —SO₂R^{a} and —SO₂NR^{b}R^{c}),
wherein R^{a} is optionally substituted C₁-C₆ alkyl, optionally substituted alkenyl, optionally substituted alkynyl, optionally substituted aryl, or optionally substituted heteroaryl;
R^{b} is H, optionally substituted C₁-C₆ alkyl, optionally substituted cycloalkyl, optionally substituted heterocycloalkyl, optionally substituted aryl, or optionally substituted heteroaryl; and
R^{c} is hydrogen or optionally substituted C₁-C₄ alkyl; or R^{b} and R^{c}, and the nitrogen to which they are attached, form an optionally substituted heterocycloalkyl group; and wherein each optionally substituted group is unsubstituted or independently substituted with one or more, such as one, two, or three, substituents independently chosen from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl or heterocycloalkyl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), or —NHSO₂(C₁-C₄ haloalkyl); and
wherein optionally substituted acyl, optionally substituted alkoxycarbonyl, sulfinyl and sulfonyl are as defined herein.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxy benzoic acid. The person skilled in the art is familiar with reaction conditions for carrying out the N-oxidation.

Compounds described herein include, but are not limited to, their optical isomers, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds include Z- and E- forms (or cis- and trans- forms) of compounds with carbon-carbon double bonds. Where compounds described herein exist in various tautomeric forms, the term “compound” is intended to include all tautomeric forms of the compound.

Compounds of Formula I also include crystalline and amorphous forms of those compounds, including, for example, polymorphs, pseudopolymorphs, solvates (including hydrates), unsolvated polymorphs (including anhydrates), conformational polymorphs, and amorphous forms of the compounds, as well as mixtures thereof. “Crystalline form,” “polymorph,” and “novel form” may be used interchangeably herein, and are meant to include all crystalline and amorphous forms of the compound, including, for example, polymorphs, pseudopolymorphs, solvates (including hydrates), unsolvated poly morphs (including anhydrates), conformational poly morphs, and amorphous forms, as well as mixtures thereof, unless a particular crystalline or amorphous form is referred to. Similarly, “pharmaceutically acceptable forms” of compounds of Formula I also include crystalline and amorphous forms of those compounds, including, for example, polymorphs, pseudopolymorphs, solvates (including hydrates), unsolvated polymorphs (including anhydrates), conformational polymorphs, and amorphous forms of the pharmaceutically acceptable salts, as well as mixtures thereof.

A “solvate” is formed by the interaction of a solvent and a compound. The term “compound” is intended to include solvates of compounds. Similarly, “pharmaceutically acceptable salts” includes solvates of pharmaceutically acceptable salts. Suitable solvates are pharmaceutically acceptable solvates, such as hydrates, including monohydrates and hemi-hydrates.

Compounds of Formula I also include other pharmaceutically acceptable forms of the recited compounds, including chelates, non-covalent complexes, prodrugs, and mixtures thereof.

A “chelate” is formed by the coordination of a compound to a metal ion at two (or more) points. The term “compound” is intended to include chelates of compounds. Similarly, “pharmaceutically acceptable salts” includes chelates of pharmaceutically acceptable salts.

A “non-covalent complex” is formed by the interaction of a compound and another molecule wherein a covalent bond is not formed between the compound and the molecule For example, complexation can occur through van der Waals interactions, hydrogen bonding, and electrostatic interactions (also called ionic bonding). Such non-covalent complexes are included in the term “compound”. Similarly, pharmaceutically acceptable salts include “non-covalent complexes” of pharmaceutically acceptable salts.

The term “hydrogen bond” refers to a form of association between an electronegative atom (also known as a hydrogen bond acceptor) and a hydrogen atom attached to a second, relatively electronegative atom (also known as a hydrogen bond donor). Suitable hydrogen bond donor and acceptors are well understood in medicinal chemistry.

“Hydrogen bond acceptor” refers to a group comprising an oxygen or nitrogen, such as an oxygen or nitrogen that is sp² -hybridized, an ether oxygen, or the oxygen of a sulfoxide or N-oxide.

The term “hydrogen bond donor” refers to an oxygen, nitrogen, or heteroaromatic carbon that bears a hydrogen. group containing a ring nitrogen or a heteroaryl group containing a ring nitrogen.

The compounds disclosed herein can be used in different enriched isotopic forms, e.g., enriched in the content of ²H, ³H, ¹¹C, ¹³C and/or ¹⁴C. In one particular embodiment, the compound is deuterated at least one position. Such deuterated forms can be made by the procedure described in U.S. Pat. Nos. 5.846.514 and 6,334.997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C .: Editor. Recent Advances in the Synthesis and Applications of Radiolabeled Compounds for Drug Discovery and Development. [In: Curr., Pharm. Des., 2000; 6(10)] 2000. 110 pp: George W .; Varma, Rajender S. The Synthesis of Radiolabeled Compounds via Organometallic Intermediates. Tetrahedron, 1989, 45(21), 6601-21; and Evans. E. Anthony. Synthesis of radiolabeled compounds, J. Radioanal. Chem., 1981, 64(1-2), 9-32.

“Pharmaceutically acceptable salts” include, but are not limited to salts with inorganic acids, such as hydrochlorate, carbonate, phosphate, hydrogenphosphate, diphosphate, hydrobromate, sulfate, sulfinate, nitrate, and like salts; as well as salts with an organic acid, such as malate, malonate, maleate, fumarate, tartrate, succinate, citrate, acetate, lactate, gluconate, methanesulfonate, Tris (hydroxymethyl-aminomethane), p-toluenesulfonate, priopionate, 2-hydroxyethylsulfonate, benzoate, salicylate, stearate, oxalate, pamoate, and alkanoate such as acetate, HOOC—(CH₂)ₙ—COOH where n is 0-4, and like salts. Other salts include sulfate, methasulfonate, bromide, trifluoracetate, picrate, sorbate, benzilate, salicilate, nitrate, phthalate or morpholine. Pharmaceutically acceptable cations include, but are not limited to sodium, potassium, calcium, aluminum, lithium, and ammonium.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

“Prodrugs” described herein include any compound that becomes a compound of Formula I when administered to a subject, e.g., upon metabolic processing of the prodrug. Similarly, “pharmaceutically acceptable salts” includes “prodrugs” of pharmaceutically acceptable salts. Examples of prodrugs include derivatives of functional groups, such as a carboxylic acid group, in the compounds of Formula I. Exemplary prodrugs of a carboxylic acid group include, but are not limited to, carboxylic acid esters such as alkyl esters, hydroxyalkyl esters, arylalkyl esters, and aryloxyalkyl esters. Other exemplary prodrugs include lower alkyl esters such as ethyl ester, acyloxyalkyl esters such as pivaloyloxymethyl (POM), glycosides, and ascorbic acid derivatives.

Other exemplary prodrugs include amides of carboxylic acids. Exemplary amide prodrugs include metabolically labile amides that are formed, for example, with an amine and a carboxylic acid. Exemplary amines include NH₂, primary, and secondary amines such as NHR^{x}, and NR^{x}R^{y}, wherein R^{x} is hydrogen, (C₁-C₁₈)-alkyl, (C₃-C₇)-cycloalkyl, (C₃-C₇)-cycloalkyl-(C₁-C₄)-alkyl-, (C₆-C₁₄)-aryl which is unsubstituted or substituted by a residue (C₁-C₂)-alkyl, (C₁-C₂)-alkoxy, fluoro, or chloro; heteroaryl-, (C₆-C₁₄)-aryl-(C₁-C₄)-alkyl- where aryl is unsubstituted or substituted by a residue (C₁-C₂)-alkyl, (C₁-C₂)-alkoxy, fluoro, or chloro; or heteroaryl-(C₁-C₄)-alkyl- and in which R^{y} has the meanings indicated for R^{x} with the exception of hydrogen or wherein R^{x} and R^{y}, together with the nitrogen to which they are bound, form an optionally substituted 4- to 7-membered heterocycloalkyl ring which optionally includes one or two additional heteroatoms chosen from nitrogen, oxygen, and sulfur. A discussion of prodrugs is provided in T. Higuchi and V. Stella, Pro-drugs as Novel Delivery Systems, Vol. 14 of the A.C.S. Symposium Series, in Edward B. Roche, ed., Bioreversible Carriers in Drug Design, American Pharmaceutical Association and Pergamon Press, 1987, and in Design of Prodrugs, ed. H. Bundgaard, Elsevier, 1985.

As used herein, the terms “group”, “radical” or “fragment” are synonymous and are intended to indicate functional groups or fragments of molecules attachable to a bond or other fragments of molecules

As used herein, the term “leaving group” refers to the meaning conventionally associated with it in synthetic organic chemistry, i.e., an atom or group displaceable under nucleophilic displacement conditions. Examples of leaving groups include, but are not limited to, dimethylhydroxylamino (e.g. Weinreb amide), halogen, alkane- or arylsulfonyloxyl, such as methanesulfonyloxy, ethanesulfonyloxy, thiomethyl, benzenesulfonyloxy, tosyloxy, and thienyloxy, dihalophosphinoyloxy, optionally substituted benzyloxy, isopropyloxy, acyloxy, and the like.

As used herein, the term “protective group” or “protecting group” refers to a group which selectively blocks one reactive site in a multifunctional compound such that a chemical reaction can be carried out selectively at another unprotected reactive site in the meaning conventionally associated with it in synthetic chemistry Certain processes of this invention rely upon the protective groups to block certain reactive sites present in the reactants. Examples of protecting groups can be found in Wuts et al., Green's Protective Groups in Organic Synthesis, (J. Wiley, 4th ed. 2006).

As used herein, the term “deprotection” or “deprotecting” refers to a process by which a protective group is removed after a selective reaction is completed. Certain protective groups may be preferred over others due to their convenience or relative ease of removal. Without being limiting, deprotecting reagents for protected amino or anilino group include strong acid such as trifluoroacetic acid (TFA), concentrated HCl, H₂SO₄, or HBr, and the like.

As used herein, “modulation” refers to a change in activity as a direct or indirect response to the presence of a chemical entity as described herein, relative to the activity of in the absence of the chemical entity. The change may be an increase in activity or a decrease in activity, and may be due to the direct interaction of the compound with the a target or due to the interaction of the compound with one or more other factors that in turn affect the target's activity. For example, the presence of the chemical entity may, for example, increase or decrease the target activity by directly binding to the target, by causing (directly or indirectly) another factor to increase or decrease the target activity, or by (directly or indirectly) increasing or decreasing the amount of target present in the cell or organism.

As used herein, “active agent” is used to indicate a chemical entity which has biological activity. In certain embodiments, an “active agent” is a compound having pharmaceutical utility. For example an active agent may be an anti-cancer therapeutic.

As used herein, “significant” refers to any detectable change that is statistically significant in a standard parametric test of statistical significance such as Student's T-test, where p<0.05.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, “therapeutically effective amount” of a chemical entity described herein refers to an amount effective, when administered to a human or non-human subject, to provide a therapeutic benefit such as amelioration of symptoms, slowing of disease progression, or prevention of disease.

“Treating” or “treatment” encompasses administration of at least one compound of Formula 1, or a pharmaceutically acceptable salt thereof, to a mammalian subject, particularly a human subject, in need of such an administration and includes (i) arresting the development of clinical symptoms of the disease, such as cancer, (ii) bringing about a regression in the clinical symptoms of the disease, such as cancer, and/or (iii) prophylactic treatment for preventing the onset of the disease, such as cancer.

As used herein, “cancer” refers to all types of cancer or neoplasm or malignant tumors found in mammals, including carcinomas and sarcomas. Examples of cancer are cancer of the brain, breast, cervix, colon, head & neck, kidney, lung, non-small cell lung, melanoma, mesothelioma, ovary, sarcoma, stomach, uterus and Medulloblastoma.

As used herein, “subject” refers to a mammal that has been or will be the object of treatment, observation or experiment. The methods described herein can be useful in both human therapy and veterinary applications. In some embodiments, the subject is a human.

The term “mammal” is intended to have its standard meaning, and encompasses humans, dogs, cats, sheep, and cows, for example.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a”, “or”, and “the” include plural referents unless the context clearly dictates otherwise.

### Methods of Treating a Cancer

The present invention provides methods of treating a cancer in an individual (such as a human individual) comprising administering to the individual an effective amount of a compound of Formula I:

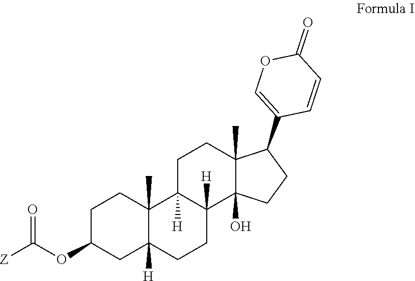

or a pharmaceutically acceptable salt thereof, wherein:
Z is —OR¹ or —NR²R³;
R¹ is optionally substituted alkyl, optionally substituted cycloalkyl, optionally substituted heterocycloalkyl, optionally substituted aryl, or optionally substituted heteroaryl; R² is hydrogen, optionally substituted alkyl, optionally substituted cycloalkyl, optionally substituted heterocycloalkyl, optionally substituted aryl, or optionally substituted heteroaryl; and R³ is optionally substituted alkyl, optionally substituted cycloalkyl, optionally substituted heterocycloalkyl, optionally substituted aryl, or optionally substituted heteroaryl, or R² and R³ are taken together with the nitrogen atom to which they are attached to form an optionally substituted heterocycloalkyl,
wherein the compound is administered at least once a week for at least two weeks (including for example at least any of 3, 4, 5, or 6 weeks).

In some embodiments, the compound of Formula I or pharmaceutically acceptable salt thereof is administered at least once a week for two weeks followed by one week of rest during a three week cycle. In some embodiments, the compound is administered at least once a week for three weeks followed by one week of rest during a four week cycle. In some embodiments, the compound of Formula I or pharmaceutically acceptable salt thereof is administered intravenously, for example by infusion for at least about 30 minutes (such as at least about any of 1, 2, 4, 6, 8, 10, 12, or 24 hours).

In some embodiments, there is provided a method of treating cancer in an individual (such as human), comprising administering to the individual an effective amount of a compound of Formula I or a pharmaceutically acceptable salt thereof, wherein the compound of Formula I or pharmaceutically acceptable salt thereof is administered daily for at least two days during a week. In some embodiments, there is provided a method of treating cancer in an individual (such as human), comprising administering to the individual an effective amount of a compound of Formula I or a pharmaceutically acceptable salt thereof, wherein the compound is administered daily for at least three days during a week. In some embodiments, there is provided a method of treating cancer in an individual (such as human), comprising administering to the individual an effective amount of a compound of Formula I or a pharmaceutically acceptable salt thereof, wherein the compound is administered daily for at least three days during a week for at least two weeks. In some embodiments, there is provided a method of treating cancer in an individual (such as human), comprising administering to the individual an effective amount of a compound of Formula I or a pharmaceutically acceptable salt thereof, wherein the compound is administered daily for at least three days during a week for at least three weeks. In some embodiments, the compound of Formula I or pharmaceutically acceptable salt thereof is administered at least on three consecutive days of a week. In some embodiments, the compound of Formula 1 or pharmaceutically acceptable salt thereof is administered on every other day during a week. In some embodiments, the compound of Formula I or pharmaceutically acceptable salt thereof is administered on days 1, 2, 3 of a week for at least two weeks (for example for two weeks in a three week cycle or three weeks in a four week cycle). In some embodiments, the compound of Formula I or pharmaceutically acceptable salt thereof is administered on days 2, 3, 4 of a week for at least two weeks (for example for two weeks in a three week cycle or three weeks in a four week cycle). In some embodiments, the compound of Formula I or pharmaceutically acceptable salt thereof is administered on days 3, 4, 5 of a week for at least two weeks (for example for two weeks in a three week cycle or three weeks in a four week cycle). In some embodiments, the compound of Formula I or pharmaceutically acceptable salt thereof is administered on days 4, 5, 6 of a week for at least two weeks (for example for two weeks in a three week cycle or three weeks in a four week cycle). In some embodiments, the compound of Formula I or pharmaceutically acceptable salt thereof is administered on days 5, 6, 7 of a week for at least two weeks (for example for two weeks in a three week cycle or three weeks in a four week cycle). In some embodiments, the compound of Formula I or pharmaceutically acceptable salt thereof is administered on days 1, 3, 5 of a week for at least two weeks (for example for two weeks in a three week cycle or three weeks in a four week cycle). In some embodiments, the compound of Formula I or pharmaceutically acceptable salt thereof is administered on days 2, 4, 6 of a week for at least two weeks (for example for two weeks in a three week cycle or three weeks in a four week cycle). In some embodiments, the compound of Formula I or pharmaceutically acceptable salt thereof is administered on days 3, 5, 7 of a week for at least two weeks (for example for two weeks in a three week cycle or three weeks in a four week cycle). In some embodiments, the compound of Formula I or pharmaceutically acceptable salt thereof is administered daily on days 1-14 of a three week cycle. In some embodiments, the compound of Formula I or pharmaceutically acceptable salt thereof is administered intravenously, for example by infusion for at least about 30 minutes (such as at least about any of 1, 2, 4, 6, 8, 10, 12, or 24 hours).

Other dosing regimens include, but are not limited to, two consecutive days and one non-consecutive day, for example days 1, 2, and 4; days 1, 2, and 5; days 1, 2, and 6; days 1, 3, and 4; days 1, 4, and 5; days 1, 5, and 6; or days 1, 6, and 7 during the week. In some embodiments, the compound of Formula I or pharmaceutically acceptable salt thereof is administered at least once a week for at least one week out of four weeks on days 1, 2, and 3 per 28 day cycle; at least two weeks out of four weeks on days 1, 2, 3, 8, 9, and 10 or days 1, 2, 3, 15, 16, and 17 per 28 day cycle; or at least three weeks out of four weeks on days 1, 2, 3, 8, 9, 10, 15, 16, and 17 per 28 day cycle. In some embodiments, the compound of Formula I or pharmaceutically acceptable salt thereof is administered at least two times a week for at least one week out of four weeks on days 1, 2, and 3 per 28 day cycle; at least two weeks out of four weeks on days 1, 2, 3, 8, 9, and 10 or days 1, 2, 3, 15, 16, and 17 per 28 day cycle; or at least three weeks out of four weeks on days 1, 2, 3, 8, 9, 10, 15, 16, and 17 per 28 day cycle. In some embodiments, the compound of Formula 1 or pharmaceutically acceptable salt thereof is administered at least three times a week for at least one week out of four weeks on days 1, 2, and 3 per 28 day cycle; at least two weeks out of four weeks on days 1, 2, 3, 8, 9, and 10 or days 1, 2, 3, 15, 16, and 17 per 28 day cycle; or at least three weeks out of four weeks on days 1, 2, 3, 8, 9, 10, 15, 16, and 17 per 28 day cycle. In some embodiments, the compound of Formula I or pharmaceutically acceptable salt thereof is administered at least three times a week for at least three weeks out of four weeks on days 1, 2, 3, 8, 9, 10, 15, 16, and 17 per 28 day cycle. In some embodiments, the compound of Formula I or pharmaceutically acceptable salt thereof is administered three times a week for three weeks (on days 1, 2, 3, 8, 9, 10, 15, 16, and 17) followed by one week of rest.

In some embodiments, the composition is administered at least once, at least twice, or at least three times per day. In some embodiments, the intervals between each administration are not more than about 7 days, not more than about 6 days, not more than about 5 days, not more than about 4 days, not more than about 3 days, not more than about 2 days, or not more than about 1 day. In some embodiments, there is no break in the dosing schedule. In some embodiments, the compound of Formula I or pharmaceutically acceptable salt thereof is administered continuously (e.g., by infusion) for at least about 2, 3, 4, 5, 6, 7, 8, 9, 10, 11, 12, 13, or 14 days.

The treatment method can be carried out over an extended period of time, such as from about a month up to about seven years. In some embodiments, the treatment method is carried out over a period of at least about any of 2, 3, 4, 5, 6, 7, 8, 9, 10, 11, 12, 18, 24, 30, 36, 48, 60, 72, or 84 months. In some embodiments, the treatment method is carried out for at least about 1, 2, 4, 6, 8, 10, or more dosing cycles.

In some embodiments, the treatment method is carried out over a period of 1 or more treatment cycles, wherein a treatment cycle is defined as 28 days, composed of 21 days of treatment and 7 days of rest. In some embodiments, the treatment method is carried out over a period of 1 treatment cycle, 2 treatment cycles, 3 treatment cycles, or 4 treatment cycles.

In some embodiments according to any of the methods described above, the compound of Formula 1 or pharmaceutically acceptable salt thereof is administered at the dose range of about 0.2 mg/m² to about 2 mg/m², including for example any of about 0.2 mg/m² to about 1.2 mg/m², about 0.4 mg/m² to about 1.2 mg/m², or about 0.4 mg/m² to about 1 mg/m². In some embodiments, the compound of Formula I or pharmaceutically acceptable salt thereof is administered in the dosage range of about 0.1 mg/m² to about 5 mg/m² (including for example about 0.15 mg/m² to about 3 mg/m² or about 0.2 mg/m² to about 2 mg/m².) Exemplary amounts of the compound of Formula I or pharmaceutically acceptable salt thereof to be administered include, but are not limited to, at least about (including about) any of 0.1 mg/m², 0.2 mg/m², 0.3 mg/m², 0.4 mg/m², 0.5 mg/m², 0.6 mg/m², 0.7 mg/m², 0.8 mg/m², 0.9 mg/m², 1 mg/m², 1.1 mg/m², 1.2 mg/m², 1.3 mg/m², 1.4 mg/m², 1.5 mg/m², 1.6 mg/m², 1.7 mg/m², 1.8 mg/m², 1.9 mg/m², 2 mg/m², 2.2 mg/m², 2.5 mg/m², 3 mg/m², 4 mg/m², and 5 mg/m². In some embodiments, the amount of the compound of Formula I or pharmaceutically acceptable salt thereof to be administered is not more than about 5 mg/m², not more than about 4 mg/m², not more than about 3 mg/m², not more than about 2.5 mg/m², not more than about 2.2 mg/m², not more than about 2 mg/m², not more than about 1.9 mg/m², not more than about 1.8 mg/m², not more than about 1.7 mg/m², not more than about 1.6 mg/m², not more than about 1.5 mg/m², not more than about 1.4 mg/m², not more than about 1.3 mg/m², not more than about 1.2 mg/m², not more than about 1.1 mg/mm², not more than about 1 mg/m², not more than about 0.9 mg/m², not more than about 0.8 mg/mm², not more than about 0.7 mg/m², not more than about 0.6 mg/m², not more than about 0.5 mg/m², not more than about 0,4 mg/m², not more than about 0.3 mg/m², not more than about 0.2 mg/m², or not more than about 0.1 mg/m². In some embodiments, the amount of the compound to be administered is about 0.1 mg/m² to about 5 mg/m², about 0.15 mg/m² to about 3 mg/m², about 0.2 mg/m² to about 2 mg/m², about 0.6 mg/m² to about 1.6 mg/m², or about 0.8 mg/m² to about 1.2 mg/m².

In some embodiments according to any of the methods described above, the compound of Formula I or pharmaceutically acceptable salt thereof is administered at the dose range of about 0.02 mg/m² to about 2 mg/m², including for example any of about 0.02 mg/m² to about 1.2 mg/m², about 0.04 mg/m² to about 1.2 mg/m², or about 0.08 mg/m² to about 1 mg/m². Exemplary amounts of the compound of Formula I or pharmaceutically acceptable salt thereof to be administered include, but are not limited to, at least about (including about) any of 0.1 mg/m², 0.15 mg/m², 0.25 mg/m², 0.35 mg/m², 0.55 mg/m², 0.8 mg/m², and 1 mg/m².

In some embodiments, the total dose of the compound of Formula I or pharmaceutically acceptable salt thereof to be administered to the individual per week is at least about 0. 1 mg/m², about 0.2 mg/m², about 0.3 mg/m², about 0.4 mg/m², about 0.5 mg/m², about 0.6 mg/m², about 0.7 mg/m², about 0.8 mg/m², about 0.9 mg/m², or about 1 mg/m². In some embodiments, the total dose of the compound of Formula I or pharmaceutically acceptable salt thereof to be administered to the individual per week is not more than about 1 mg/m², not more than about 0.9 mg/m², not more than about 0.8 mg/m², not more than about 0.7 mg/m², not more than about 0.6 mg/m², not more than about 0.5 mg/m², not more than about 0.4 mg/m², not more than about 0.3 mg/m², not more than about 0.2 mg/m², or not more than about 0.1 mg/m². In some embodiments, the total dose of the compound of Formula I or pharmaceutically acceptable salt thereof to be administered to the individual per week is about 0.1 mg/m² to about 1 mg/m², about 0.2 mg/m² to about 0.7 mg/m², or about 0.3 mg/m² to about 0.5 mg/m². In some embodiments, the total dose per administration cycle is at least about 2 mg/m² to about 6 mg/m².

The compound of Formula I or pharmaceutically acceptable salt thereof can be provided in vials at the concentration of at least about 0.01 mg/mL, at least about 0.05 mg/mL, at least about 0.06 mg/mL, at least about 0.07 mg/mL, at least about 0.08 mg/mL, at least about 0.09 mg/mL, at least about 0.1 mg/mL, at least about 0.11 mg/mL, at least about 0.12 mg/mL, at least about 0.13 mg/mL, at least about 0.14 mg/mL, at least about 0.15 mg/mL, at least about 0.2 mg/mL, at least about 0.3 mg/mL, at least about 0.4 mg/mL, at least about 0.5 mg/mL, at least about 0.6 mg/mL, at least about 0.7 mg/mL, at least about 0.8 mg/mL, at least about 0.9 mg/mL, at least about 1 mg/mL, at least about 2.5 mg/mL, or at least about 5 mg/mL. In some embodiments, the compound of Formula I or pharmaceutically acceptable salt thereof can be provided in vials at the concentration of not more than about 5 mg/mL, not more than about 2.5 mg/mL, not more than about 1 mg/mL, not more than about 0.9 mg/mL, not more than about 0.8 mg/mL, not more than about 0.7 mg/mL, not more than about 0.6 mg/mL, not more than about 0.5 mg/mL, not more than about 0.4 mg/mL, not more than about 0.3 mg/mL, not more than about 0.2 mg/mL, not more than about 0. 1 mg/mL, not more than about 0.00 mg/mL, not more than about 0.08 mg/mL, not more than about 0.07 mg/mL, not more than about 0.06 mg/mL, not more than about 0.05 mg/mL, or not more than about 0.01 mg/mL. In some embodiments, the compound of Formula I or pharmaceutically acceptable salt thereof can be provided in vials at the concentration of about 0.01 mg/mL, to about 5 mg/mL, about 0.05 mg/mL, to about 2.5 mg/mL, about 0.06 mg/mL, to about 1 mg/mL, about 0.07 mg/mL to about 0.9 mg/mL, about 0.08 mg/mL to about 0.5 mg/mL, or about 0.09 mg/mL to about 0.11 mg/mL.

The compound of Formula I or pharmaceutically acceptable salt thereof can be diluted prior to administration, for example to the concentration of at least about 0.0001 mg/mL, at least about 0.0005 mg/mL, at least about 0.001 mg/mL, at least about 0.0015 mg/mL, at least about 0.0017 mg/mL, at least about 0.0018 mg/mL, at least about 0.0019 mg/mL, at least about 0.002 mg/mL, at least about 0.0021 mg/mL, at least about 0.0022 mg/mL, at least about 0.0023 mg/mL, at least about 0.0025 mg/mL, at least about 0.003 mg/mL, at least about 0.004 mg/mL, at least about 0.005 mg/mL, at least about 0.006 mg/mL, at least about 0.007 mg/mL, at least about 0.008 mg/mL, at least about 0.009 mg/mL, at least about 0.01 mg/mL, at least about 0.011 mg/mL, at least about 0.012 mg/mL, at least about 0.013 mg/mL, at least about 0.015 mg/mL, at least about 0.02 mg /mL, at least about 0.05 mg/mL, or at least about 0.1 mg/mL. In some embodiments, the compound of Formula 1 or pharmaceutically acceptable salt thereof can be diluted prior to administration, for example to the concentration of not more than about 0.1 mg/mL, not more than about 0.05 mg/mL, not more than about 0.02 mg/mL, not more than about 0.015 mg/mL, not more than about 0.013 mg/mL, not more than about 0.012 mg/mL, not more than about 0.011 mg/mL, not more than about 0.01 mg/mL, not more than about 0.009 mg/mL, not more than about 0.008 mg/mL, not more than about 0.007 mg/mL, not more than about 0.006 mg/mL, not more than about 0.005 mg/mL, not more than about 0.004 mg/mL, not more than about 0.003 mg/mL, not more than about 0.0025 mg/mL, not more than about 0.0023 mg/mL, not more than about 0.0022 mg/mL, not more than about 0.0021 mg/mL, not more than about 0.002 mg/mL, not more than about 0.0019 mg/mL, not more than about 0.0018 mg/mL, not more than about 0.0017 mg/mL, not more than about 0.0015 mg/mL, not more than about 0.001 mg/mL, not more than about 0.0005 mg/mL, or not more than about 0.0001 mg/mL. In some embodiments, the compound of Formula I or pharmaceutically acceptable salt thereof can be diluted prior to administration, for example to the concentration of about 0.0001 mg/mL to about 0.1 mg/mL, about 0.001 mg/mL to about 0.05 mg/mL, about 0.0015 mg/mL, to about 0.02 mg/mL, or about 0.002 mg/mL to about 0.01 mg/mL.

The compound of Formula I or pharmaceutically acceptable salt thereof described herein can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In some embodiments, the compound of Formula I or pharmaceutically acceptable salt thereof is administered intravenously. In some embodiments, the compound of Formula I or pharmaceutically acceptable salt thereof is administered via infusion, for example by infusion for at least about (including about) any of 20 minutes, 30 minutes, 40 minutes, 50 minutes, or 60 minutes. In some embodiments, the compound of Formula I or pharmaceutically acceptable salt thereof is administered via infusion for at least about (including about) any of 1. 2. 4. 6, 8, 10. 12, or 24 hours. In some embodiments, the compound of Formula I or pharmaceutically acceptable salt thereof is infused over an infusion period of at least about 15 minutes, at least about 30 minutes, at least about 1 hour, at least about 2 hours, at least about 3 hours, at least about 5 hours, at least about 8 hours, at least about 12 hours, or at least about 24 hours. In some embodiments, the composition is administered over an infusion period of not more than about 24 hours, not more than about 12 hours, not more than about 8 hours, not more than about 5 hours, not more than about 3 hours, not more than about 2 hours, not more than about 1 hour, not more than about 30 minutes, or not more than about 10 minutes.

### Treatment of Cancer

The methods described herein are generally useful for treating cancer in an individual. In some embodiments, the cancer to be treated is solid tumor. In some embodiments, the cancer to be treated is liquid tumor. In some embodiments, the solid tumor is any of early stage cancer. con-metastatic cancer, primary cancer, advanced cancer, locally advanced cancer, metastatic cancer, cancer in remission, cancer in an adjuvant setting, or cancer in a neoadjuvant setting. In some embodiments, the solid tumor is localized resectable, localized unresectable, or unresectable. In some embodiments, the solid tumor is localized reseciable or borderline resectable. In some embodiments, the cancer has been refractory to prior therapy. In some embodiments, the cancer is resistant to the treatment with a chemotherapeutic agent other than the compound.

The methods provided herein may be practiced in an adjuvant setting. In some embodiments, the method is practiced in a neoadjuvant setting, i.e., the method may be carried out before the primary/definitive therapy. In some embodiments, the method is used to treat an individual who has previously been treated. In some embodiments, the individual has progressed on or failed at least one prior systemic therapy. The methods of treatment provided herein may also be used to treat an individual who has not previously been treated. In some embodiments, the method is used as a first line therapy. In some embodiments, the method is used as a second line therapy.

In some embodiments, the cancer includes, but is not limited to, colon carcinoma, colorectal cancer, liver cancer, pancreatic cancer, breast cancer, ovarian cancer, prostate cancer, thyroid cancer, oral cancer, fibrosarcoma, myxosarcoma, liposarcoma, chondrosarcoma, osteogenic sarcoma, chondroma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung cancer, bladder carcinoma, epithelial carcinoma, melanoma, leukemia, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polyeythemia vera, lymphoma (Hodgkin's disease and non-Hodgkin's disease), multiple myeloma, Waldenstrom's macroglobulinemia, and heavy chain disease. In some embodiments, the cancer is selected from the group consisting of colorectal cancer, liver cancer, lung cancer, breast cancer, pancreatic cancer, prostate cancer, and oral cancer. In some embodiments, the cancer is selected from the group consisting of liver cancer, colorectal cancer, and lung cancer.

In some embodiments, the cancer is selected from the group consisting of colorectal cancer, breast cancer, liver cancer, gastric cancer, and non-small cell lung cancer.

In some embodiments, the cancer to be treated is lung cancer. In some embodiments, the lung cancer is a non-small cell lung cancer (NSCLC). Examples of NSCLC include, but are not limited to, large-cell carcinoma (e.g., large-cell neuroendocrine carcinoma, combined large-cell neuroendocrine carcinoma, basaloid carcinoma, lymphoepithelioma-like carcinoma, clear cell carcinoma, and large-cell carcinoma with rhabdoid phenotype), adenocarcinoma (e.g., acinar, papillary (e.g., bronchioloalveolar carcinoma, nonmucinous, mucinous, mixed mucinous and nonmucinous and indeterminate cell type), solid adenocarcinoma with mucin, adenocarcinoma with mixed subtypes, well-differentiated fetal adenocarcinoma, mucinous (colloid) adenocarcinoma, mucinous cystadenocarcinoma, signet ring adenocarcinoma, and clear cell adenocarcinoma), neuroendocrine lung tumors, and squamous cell carcinoma (e.g., papillary, clear cell, small cell, and basaloid). In some embodiments, the NSCLC is, according to TNM classifications, a stage T tumor (primary tumor), a stage N tumor (regional lymph nodes), or a stage M tumor (distant metastasis) In some embodiments, the lung cancer is a carcinoid (typical or atypical), adenosquamous carcinoma, cylindroma, or carcinoma of the salivary gland (e.g., adenoid cystic carcinoma or mucoepidermoid carcinoma). In some embodiments, the lung cancer is a carcinoma with pleomorphic, sarcomatoid, or sarcomatous elements (e.g., carcinomas with spindle and/or giant cells, spindle cell carcinoma, giant cell carcinoma, carcinosarcoma, or pulmonary blastoma). In some embodiments, the cancer is small cell lung cancer (SCLC; also called oat cell carcinoma). The small cell lung cancer may be limited-stage, extensive stage or recurrent small cell lung cancer. In some embodiments, the individual may be a human who has a gene. genetic mutation, or polymorphism suspected or shown to be associated with lung cancer (e.g., SASH1, LATS1, IGF2R, PARK2, KRAS, PTEN, Kras2, Krag, Pas1, ERCC1, XPD, IL8RA, EGFR, Ot1-AD, EPHX, MMP1, MMP2, MMP3, MMP 12, IL1 β, RAS, and/or AKT) or has one or more extra copies of a gene associated with lung cancer.

In some embodiments, the cancer to be treated is colorectal cancer. In some embodiments, the individual may be a human who has a gene, genetic mutation, or poly morphism associated with colorectal cancer (e.g., RAS, AKT, PTEN, POK, and/or EGFR) or has one or more extra copies of a gene associated with colorectal cancer.

In some embodiments, the cancer to be treated is liver cancer, such as hepatocellular carcinoma (HCC). In some embodiments, the HCC is early stage HCC, non-metastatic HCC, primary HCC, advanced HCC, locally advanced HCC, metastatic HCC, HCC in remission, or recurrent HCC. In some embodiments, the HCC is localized resectable (i.e., tumors that are confined to a portion of the liver that allows for complete surgical removal), localized unresectable (i.e., the localized tumors may be unresectable because crucial blood vessel structures are involved or because the liver is impaired), or unresectable (i.e., the tumors involve all lobes of the liver and/or has spread to involve other organs (e.g. , lung, lymph nodes, bone)) In some embodiments, the HCC is, according to TNM classifications, a stage I tumor (single tumor without vascular invasion), a stage II tumor (single tumor with vascular invasion, or multiple tumors, none greater than 5 cm), a stage III tumor (multiple tumors, any greater than 5 cm, or tumors involving major branch of portal or hepatic veins), a stage IV tumor (tumors with direct invasion of adjacent organs other than the gallbladder, or perforation of visceral peritoneum), N1 tumor (regional lymph node metastasis), or M1 tumor (distant metastasis). In some embodiments, the HCC is, according to AJCC (American Joint Commission on Cancer) staging criteria, stage T1, T2, T3, or T4 HCC. In some embodiments, the HCC is any one of liver cell carcinomas, fibrolamellar variants of HCC, or mixed hepatocellular cholangiocarcinomas. In some embodiments, the individual may be a human who has a gene, genetic mutation, or polymorphism associated with hepatocellular carcinoma (e.g., CCND2, RAD23B, GRP78, CEP164, MDM2, and/or ALDH2) or has one or more extra copies of a gene associated with hepatocellular carcinoma.

In some embodiments, the cancer to be treated is breast cancer. In some embodiments, the compound of Formula I or pharmaceutically acceptable salt thereof is administered intravenously. Breast cancer described herein can include early stage breast cancer, non-metastatic breast cancer, stage IV breast cancer, locally advanced breast cancer, metastatic breast cancer, hormone receptor positive metastatic breast cancer, breast cancer in remission, breast cancer in an adjuvant setting, ductal carcinoma in situ (DCIS), invasive ductal carcinoma (IDC), and breast cancer in a neoadjuvant setting. In some embodiments, the breast cancer is hormone receptor positive metastatic breast cancer. In some embodiments, the breast cancer (which may be HER2 positive or HER2 negative) is advanced breast cancer. In some embodiments, the breast cancer is ductal carcinoma in situ. In some embodiments, the individual may be a human who has a gene, genetic mutation, or polymorphism associated with breast cancer (e.g., BRCA), BRCA2, ATM, CHEK2, RAD51, AR, DIRAS3, ERBB2, TP53, AKT, PTEN, and/or PI3K) or has one or more extra copies of a gene (e.g., one or more extra copies of the HER2 gene) associated with breast cancer.

In some embodiments, the cancer to be treated is pancreatic cancer. Pancreatic cancer that can be treated includes, but is not limited to, exocrine pancreatic cancer and endocrine pancreatic cancer. The exocrine pancreatic cancer includes, but is not limited to, adenocarcinomas, acinar cell carcinomas, adenosquamous carcinomas, colloid carcinomas, undifferentiated carcinomas with osteoclast-like giant cells, hepatoid carcinomas, intraductal papillary-mucinous neoplasms, mucinous cystic neoplasms, pancreatoblastomas, serous cystadenomas, signet ring cell carcinomas, solid and pseuodpapillary tumors, pancreatic ductal carcinomas, and undifferentiated carcinomas. In some embodiments, the exocrine pancreatic cancer is pancreatic ductal carcinoma. The endocrine pancreatic cancer includes, but is not limited to, insulinomas and glucagonomas.

In some embodiments, the cancer to be treated is prostate cancer. In some embodiments, the prostate cancer is an adenocarcinoma. In some embodiments, the prostate cancer is a sarcoma, neuroendocrine tumor, small cell cancer, ductal cancer, or a lymphoma. In some embodiments, the prostate cancer is at any of the four stages, A, B, C, or D, according to the Jewett staging system. In some embodiments, the prostate cancer is stage A prostate cancer (e.g., the cancer cannot be felt during a rectal exam). In some embodiments, the prostate cancer is stage B prostate cancer (e.g., the tumor involves more tissue within the prostate, and can be felt during a rectal exam, or is found with a biopsy that is done because of a high PSA level). In some embodiments, the prostate cancer is stage C prostate cancer (e.g., the cancer has spread outside the prostate to nearby tissues). In some embodiments, the prostate cancer is stage D prostate cancer. In some embodiments, the prostate cancer is androgen independent prostate cancer (AIPC). In some embodiments, the prostate cancer is androgen dependent prostate cancer. In some embodiments, the prostate cancer is refractory to hormone therapy.

The methods described herein are useful for various aspects of cancer treatment. For example, in some embodiments, there is provided a method of treating cancer (such as liver cancer, lung cancer, or colorectal cancer) in an individual, comprising administering to the individual an effective amount of a compound of Formula I (such as any one of the compounds listed in Table 1 below) or a pharmaceutically acceptable salt thereof, wherein the compound is administered at least once a week for at least two weeks In some embodiments, the compound is administered at least once a week for two weeks followed by one week of rest during a three week cycle. In some embodiments, the compound is administered at least once a week for three weeks followed by one week of rest during a four week cycle. In some embodiments, the compound is administered daily for at least three days during a week for at least two weeks (such as three weeks). In some embodiments, the compound is administered at least on three consecutive days of a week. In some embodiments, the compound is administered on every other day during a week. In some embodiments, the compound is administered daily on days 1-14 of a three week cycle. In some embodiments, the compound is administered intravenously, for example by infusion for at least about 30 minutes (such as at least about any of 1, 2, 4, 6. 8. 10. 12, or 24 hours).

In some embodiments, there is provided a method of inhibiting cancer cell proliferation (such as tumor growth) in an individual, comprising administering to the individual an effective amount of a compound of Formula I (such as any one of the compounds listed in Table 1 below) or a pharmaceutically acceptable salt thereof, wherein the compound is administered at least once a week for at least two weeks. In some embodiments, at least about 10% (including for example at least about any of 20%, 30%, 40%, 60%, 70%, 80%, 90%, or 100%) cell proliferation is inhibited.

In some embodiments, there is provided a method of inhibiting tumor metastasis in an individual, comprising administering to the individual an effective amount of a compound of Formula I (such as any one of the compounds listed in Table 1 below) or a pharmaceutically acceptable salt thereof, wherein the compound is administered at least once a week for at least two weeks. In some embodiments, at least about 10% (including for example at least about any of 20%, 30%, 40%, 60%, 70%, 80%, 90%, or 100%) metastasis is inhibited. In some embodiments, method of inhibiting metastasis to lymph node is provided. In some embodiments, method of inhibiting metastasis to the lung is provided.

In some embodiments, there is provided a method of reducing (such as eradicating) pre-existing tumor metastasis (such as pulmonary metastasis or metastasis to the lymph node) in an individual, comprising administering to the individual an effective amount of a compound of Formula I (such as any one of the compounds listed in Table 1 below) or a pharmaceutically acceptable salt thereof, wherein the compound is administered at least once a week for at least two weeks. In some embodiments, at least about 10% (including for example at least about any of 20%, 30%, 40%, 60%, 70%, 80%, 90%, or 100%) metastasis is reduced. In some embodiments, method of reducing metastasis to lymph node is provided.

In some embodiments, there is provided a method of reducing incidence or burden of preexisting tumor metastasis (such as pulmonary metastasis or metastasis to the lymph node) in an individual, comprising administering to the individual an effective amount of a compound of Formula I (such as any one of the compounds listed in Table 1 below) or a pharmaceutically acceptable salt thereof, wherein the compound is administered at least once a week for at least two weeks.

In some embodiments, there is provided a method of reducing tumor size in an individual, comprising administering to the individual an effective amount of a compound of Formula I (such as any one of the compounds listed in Table 1 below) or a pharmaceutically acceptable salt thereof, wherein the compound is administered at least once a week for at least two weeks. In some embodiments, the tumor size is reduced at least about 10% (including for example at least about any of 20%, 30%, 40%. 60%, 70%, 80%, 90%, or 100%).

In some embodiments, there is provided a method of prolonging time to disease progression of a cancer in an individual, comprising administering to the individual an effective amount of a compound of Formula I (such as any one of the compounds listed in Table 1 below) or a pharmaceutically acceptable salt thereof, wherein the compound is administered at least once a week for at least two weeks In some embodiments, the method prolongs the time to disease progression by at least any of 1, 2, 3, 4, 5, 6, 7, 8, 9, 10, 11, or 12 weeks.

In some embodiments, there is provided a method of prolonging survival of an individual having cancer, comprising administering to the individual an effective amount of a compound of Formula I (such as any one of the compounds listed in Table 1 below) or a pharmaceutically acceptable salt thereof, wherein the compound is administered at least once a week for at least two weeks. In some embodiments, the method prolongs the survival of the individual by at least any of 1, 2, 3, 4, 5, 6, 7, 8, 9, 10, 11, 12, 18, or 24 month.

In some embodiments, there is provided a method of alleviating one or more symptoms in an individual having cancer, comprising administering to the individual an effective amount of a compound of Formula I (such as any one of the compounds listed in Table 1 below) or a pharmaceutically acceptable salt thereof, wherein the compound is administered at least once a week for at least two weeks.

In some embodiments, there is provided a method of treating cancer (such as liver cancer, lung cancer, or colorectal cancer) in an individual, comprising administering (e.g., by infusion) to the individual an effective amount of a compound of Formula I (such as any one of the compounds listed in Table 1 below) or a pharmaceutically acceptable salt thereof, wherein the compound is administered on days 1, 2, 3 of every week for three weeks followed by one week of rest. In some embodiments, the composition is administered at the dosage of about 0.2 to about 1.2 mg/m² (for example about any of 0.2, 0.4, 0.6, 0.8, 1, or 1.2 mg/m²). In some embodiments, the compound is administered at the concentration of about 0.001 to about 0.05 mg/mL (such as about 0.002 to about 0.01 mg/mL). In some embodiments, the compound is administered as a pharmaceutical composition comprising about 0.1M sodium acetate/acetic acid. In some embodiments, the individual has progressed on, or failed at least one prior systemic therapy. In some embodiments, the cancer is locally advanced or metastatic.

In some embodiments, there is provided a method of treating cancer (such as liver cancer, lung cancer, or colorectal cancer) in an individual, comprising administering (e.g., by infusion) to the individual an effective amount of a compound of Formula I (such as any one of the compounds listed in Table 1 below) or a pharmaceutically acceptable salt thereof, wherein the compound is administered on days 1, 2, 3 of every week for three weeks followed by one week of rest. In some embodiments, the composition is administered at the dosage of about 0.02 to about 1.2 mg/m² (for example about any of 0.02, 0.08, 0.2, 0.4, 0.6, 0.8, 1, or 1.2 mg/m²). In some embodiments, the compound is administered at the concentration of about 0.001 to about 0.05 mg/mL (such as about 0.002 to about 0.01 mg/mL). In some embodiments, the compound is administered as a pharmaceutical composition comprising about 0.1M sodium acetate/acetic acid. In some embodiments, the individual has progressed on, or failed at least one prior systemic therapy. In some embodiments, the cancer is locally advanced or metastatic.

In some embodiments, there is provided a method of treating cancer (such as liver cancer, lung cancer, or colorectal cancer) in an individual, comprising administering (e.g., by infusion) to the individual an effective amount of a compound of Formula I (such as any one of the compounds listed in Table 1 below) or a pharmaceutically acceptable salt thereof, wherein the compound is administered on day 1 of every week for three weeks followed by one week of rest. In some embodiments, the composition is administered at the dosage of about 0.2 to about 1.2 mg/m² (for example about any of 0.2, 0.4, 0.6, 0.8, 1, or 1.2 mg/m²). In some embodiments, the compound is administered at the concentration of about 0.001 to about 0.05 mg/mL (such as about 0.002 to about 0.01 mg/mL.). In some embodiments, the compound is administered as a pharmaceutical composition comprising 0.1M sodium acetate/acetic acid. In some embodiments, the individual has progressed on, or failed at least one prior systemic therapy. In some embodiments, the cancer is locally advanced or metastatic.

In some embodiments, there is provided a method of treating cancer (such as liver cancer, lung cancer, or colorectal cancer) in an individual, comprising administering (e.g., by infusion) to the individual an effective amount of a compound of Formula I (such as any one of the compounds listed in Table 1 below) or a pharmaceutically acceptable salt thereof, wherein the compound is administered on day 1 of every week for three weeks followed by one week of rest. In some embodiments, the composition is administered at the dosage of about 0.02 to about 1.2 mg/m² (for example about any of 0.02, 0.08, 0.2, 0.4, 0.6, 0.8, 1, or 1.2 mg/m²). In some embodiments, the compound is administered at the concentration of about 0.001 to about 0.05 mg/mL (such as about 0.002 to about 0.01 mg/mL). In some embodiments, the compound is administered as a pharmaceutical composition comprising 0.1M sodium acetate/acetic acid. In some embodiments, the individual has progressed on, or failed at least one prior systemic therapy. In some embodiments, the cancer is locally advanced or metastatic.

In some embodiments, there is provided a method of treating cancer (such as liver cancer, lung cancer, or colorectal cancer) in an individual, comprising administering (e.g., by infusion) to the individual an effective amount of a compound of Formula I (such as any one of the compounds listed in Table 1 below) or a pharmaceutically acceptable salt thereof, wherein the compound is administered continuously for two weeks followed by one week of rest. In some embodiments, the composition is administered at the dosage of about 0.4 to about 1.2 mg/m² (for example about any of 0.4, 0.6, 0.8, 1, or 1.2 mg/m²). In some embodiments, the compound is administered at the concentration of about 0.001 to about 0.05 mg/mL (such as about 0.002 to about 0.01 mg/mL). In some embodiments, the compound is administered as a pharmaceutical composition comprising 0.1M sodium acetate/acetic acid. In some embodiments, the individual has progressed on, or failed at least one prior systemic therapy. In some embodiments, the cancer is locally advanced or metastatic.

In some embodiments, there is provided a method of treating cancer (such as liver cancer, lung cancer, or colorectal cancer) in an individual, comprising administering (e.g., by infusion) to the individual an effective amount of a compound of Formula I (such as any one of the compounds listed in Table 1 below) or a pharmaceutically acceptable salt thereof, wherein the compound is administered continuously for two weeks followed by one week of rest. In some embodiments, the composition is administered at the dosage of about 0.04 to about 1.2 mg/m² (for example about any of 0.04, 0.08, 0.4, 0.6, 0.8, 1, or 1.2 mg/m²). In some embodiments, the compound is administered at the concentration of about 0.001 to about 0.05 mg/mL. (such as about 0.002 to about 0.01 mg/mL). In some embodiments, the compound is administered as a pharmaceutical composition comprising 0.1M sodium acetate/acetic acid. In some embodiments, the individual has progressed on, or failed at least one prior systemic therapy. In some embodiments, the cancer is locally advanced or metastatic.

### Biomarkers

In some embodiments, the levels of PI3K and/or pAKT can be used as a basis for selecting patients for treatment. The levels of PI3K and/or pAKT can be used, for example, for determining (and aiding assessment) in any one or more of the following: a) probable or likely suitability of an individual to initially receive treatment; b) probable or likely unsuitability of an individual to initially receive treatment(s); c) responsiveness to treatment; d) probable or likely suitability of an individual to continue to receive treatment; e) probable or likely unsuitability of an individual to receive treatment(s); f) adjusting dosages; and g) predicting likelihood of clinical benefits. The present application encompasses any of these methods.

In some embodiments, there is provided a method of treating cancer in an individual (such as a human individual) comprising administering (such as intravenously administering) to the individual an effective amount of a compound of Formula I, wherein the individual has a high level of PI3K, and optionally wherein the compound is administered at least once a week for at least two weeks. In some embodiments, there is provided a method of treating cancer in an individual (such as a human individual) comprising administering (such as intravenously administering) to the individual an effective amount of a compound of Formula 1, wherein the level of PI3K is used as a basis for selecting the individual for treatment, and optionally wherein the compound is administered at least once a week for at least two weeks. In some embodiments, the individual is selected for treatment if the individual has a high level of PI3K. In some embodiments, the level of PI3K is determined by immunohistochemistry method. In some embodiments, the level of the PI3K is based on protein expression level. In some embodiments, the level of the PI3K is based on mRNA level. In some embodiments, the method further comprises determining the level of the PI3K prior to the treatment. In some embodiments, the method further comprises selecting the individual for treatment based on the PI3K.

In some embodiments, there is provided a method of treating cancer in an individual (such as a human individual) comprising administering (such as intravenously administering) to the individual an effective amount of a compound of Formula 1, wherein the individual has a high level of pAKT, and optionally wherein the compound is administered at least once a week for at least two weeks. In some embodiments, there is provided a method of treating cancer in an individual (such as a human individual) comprising administering (such as intravenously administering) to the individual an effective amount of a compound of Formula I, wherein the level of pAKT is used as a basis for selecting the individual for treatment, and optionally wherein the compound is administered at least once a week for at least two weeks. In some embodiments, the individual is selected for treatment if the individual bas a high level of pAKT. In some embodiments, the level of PI3K is determined by immunohistochemistry method. In some embodiments, the method further comprises determining the level of the pAKT prior to the treatment. In some embodiments, the method further comprises selecting the individual for treatment based on the PI3K.

### Compounds of Formula (I)

The compounds used in the methods described herein is a compound of Formula I:

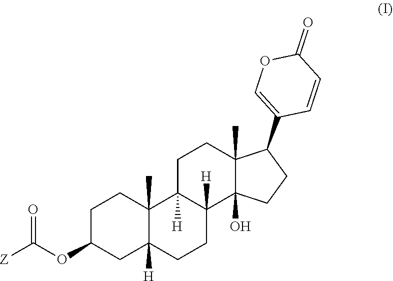

or a pharmaceutically acceptable salt thereof, wherein:
Z is —OR¹ or —NR²R³;
R¹ is optionally substituted alkyl, optionally substituted cycloalkyl, optionally substituted heterocycloalkyl, optionally substituted aryl, or optionally substituted heteroaryl; R² is hydrogen. optionally substituted alkyl, optionally substituted cycloalkyl, optionally substituted heterocycloalkyl, optionally substituted aryl, or optionally substituted heteroaryl; and R³ is optionally substituted alkyl, optionally substituted cycloalkyl, optionally substituted heterocycloalkyl, optionally substituted aryl, or optionally substituted heteroaryl, or R² and R³ are taken together with the nitrogen atom to which they are attached to form an optionally substituted heterocycloalkyl.

In some embodiments, Z is —OR¹. In some embodiments, Z is —OR¹ and R¹ is optionally substituted alkyl, optionally substituted cycloalkyl, or optionally substituted heterocycloalkyl. In some embodiments, R¹ is optionally substituted alkyl. In some embodiments, R¹ is optionally substituted ethyl. In some embodiments, R¹ is ethyl substituted by heterocycloalkyl. In some embodiments, R¹ is ethyl substituted by pyrrolidinyl (e.g., pyrrolidin-1-yl) or morpholinyl (e.g., morpholin-4-yl).

In some embodiments, Z is —NR²R³. In some embodiments, R² is hydrogen, optionally substituted alkyl, optionally substituted cycloalkyl, or optionally substituted heterocycloalkyl. In some embodiments, R² is hydrogen. In some embodiments, R³ is optionally substituted alkyl, optionally substituted cycloalkyl, or optionally substituted heterocycloalkyl. In some embodiments, R³ is optionally substituted alkyl. In some embodiments, R³ is optionally substituted ethyl or optionally substituted propyl (e.g., n-propyl). In some embodiments, R³ is ethyl substituted by heterocycloalkyl. In some embodiments, R¹ is ethyl substituted by pyrrolidinyl (e.g., pyrrolidin-1-yl) or morpholinyl (e.g., morpholin-4-yl). In some embodiments, R³ is n-propyl substituted by carboxylic acid. In some embodiments, R² is hydrogen, optionally substituted alkyl, optionally substituted cycloalkyl, or optionally substituted heterocycloalkyl, and R³ is optionally substituted alkyl, optionally substituted cycloalkyl, or optionally substituted heterocycloalkyl. In some embodiments, R² is hydrogen, and R³ is optionally substituted alkyl. In some embodiments, R² is hydrogen, and R³ is optionally substituted ethyl or optionally substituted propyl (e.g., n-propyl). In some embodiments, R²is hydrogen, and R³ is ethyl substituted by heterocycloalkyl. In some embodiments, R² is hydrogen, and R³ is ethyl substituted by pyrrolidinyl (e.g., pyrrolidin-1-yl) or morpholinyl (e.g., morpholin-4-yl). In some embodiments, R² is hydrogen, and R³ is n-propyl substituted by carboxylic acid.

In some embodiments, Z is —NR²R³, and R² and R³ are taken together with the atom to which they are attached to form a heterocycloalkyl. In some embodiments, R² and R³ are taken together with the nitrogen atom to which they are attached to form a 4- to 8-membered heterocycloalkyl, which comprises 0 or 1 additional annular heteroatom. In some embodiments, R² and R³ are taken together with the nitrogen atom to which they are attached to form a 6-membered heterocycloalkyl containing 1 additional annular nitrogen or oxygen atom. In some embodiments, R² and R³ are taken together with the nitrogen atom to which they are attached to form piperazinyl (e.g., piperazin-1-yl) or morpholinyl (e.g., morpholin-4-yl).

Representative compounds are listed in Table 1.

| Compound | | |
|---|---|---|
| No. | Structure | Chemical Name¹ |
| 1 | 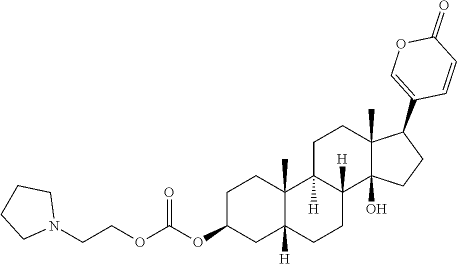 | (38,5R,8R,9S,10S,13R,14S,17R)-14- hydroxy-10.13-dimethyl-17-(2-oxo- 2H-pyran-5-yl)hexadecahydro-1H- cyclopenta[a]phenanthren-3-yl (2- (pyrrolidin-1-yl)ethyl) carbonate |
| 2 | 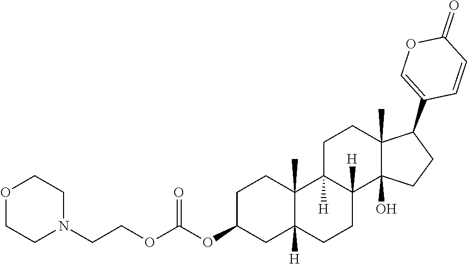 | (3S,5R,8R,9S,108,13R,14S,17R)-14- hydroxy-10,13-dimethyl-17-(2-oxo- 2H-pyran-5-yl)hexadecahydro-1H- cyclopenta[a]phenanthren-3-yl (2- morpholinoethyl) carbonate |
| 3 | 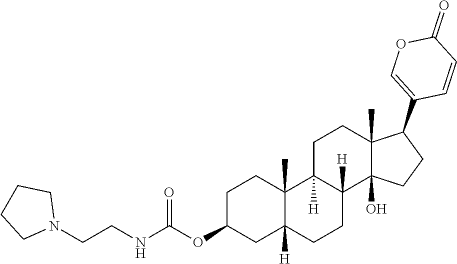 | (3S,SR,8R,9S,10S,13R,14S,17R)- 14-hydroxy-10,13-dimethyl-17-(2- oxo-2H-pyran-5-yl)hexadecahydro- 1/-cyclopenta[a]phenanthren-3-yl (2- (pyrrolidin-1-yD)ethyl)carbamate |
| 4 | 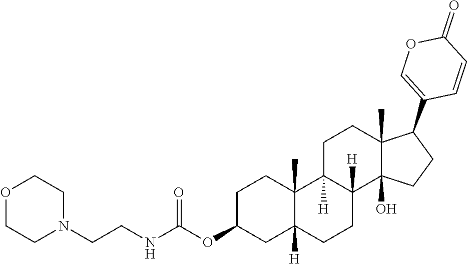 | (3S,5R,8R,9S,10S,13R,14S,17R)-14- hydroxy-10,13-dimethyl-17-(2-oxo- 2H-pyran-5-yl)hexadecahydro-1H- cyclopenta[@]phenanthren-3-yl (2- morpholinoethyl)carbamate |
| 5 | 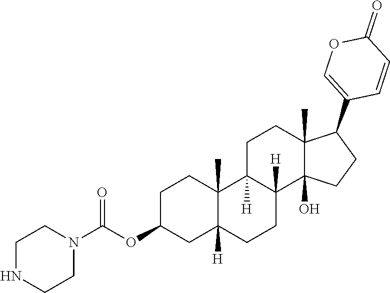 | (35,5R,8R,9S,10S,13R,145,17R)-14- hydroxy-10,13-dimethyl-17-(2-oxo- 2/-pyran-5-yl)hexadecahydro-1H- cyclopenta[a]phenanthren-3-yl piperazine-1-carboxylate |
| 6 | 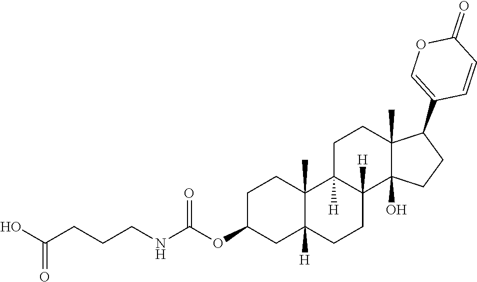 | 4- (((((3S,SR.8R,9S,10S,13R,145,17R)- 14-hydroxy~10,13-dimethyl-17-(2- oxo-2H-pyran-5-yl)hexadecahydro- 1/-cyclopenta[a]phenanthren-3- yl)oxy )carbonyl)amino)butanoic acid |
| 7 | 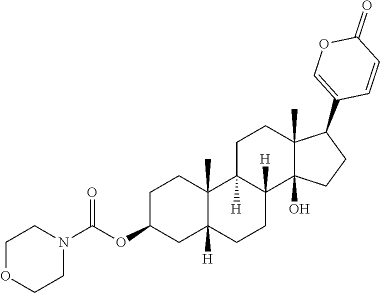 | (3S,5R,8R,9S,10S,13R,14S,17R)-14- hydroxy-10,13-dimethyl-17-(2-oxo- 2H-pyran-5-yl)hexadecahydro-1H- cyclopenta[a]phenanthren-3-yl morpholine-4-carboxylate |

¹Chemical names are generated using the ChemBioDraw ® Ultra version 16.0.0.82 (68) software.

### Pharmaceutical Compositions

The compounds described herein can be provided in a pharmaceutical composition comprising the compound and a pharmaceutically acceptable carrier. In some embodiments, the pharmaceutical composition is suitable for administration to a human In some embodiments, the pharmaceutical composition is suitable for administration to a mammal, such as, in the veterinary context, domestic pets and agricultural animals.

There are a wide variety of suitable formulations of the composition. The following formulations and methods are merely exemplary and are in no way limiting. For example, formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizing agents, and preservatives. The formulations can be presented in unit-dose or multi-dose sealed containers, such as ampules and vials, and can be stored in a freeze-dried (lyophilized) condition requiring only the addition of a sterile liquid excipient (e.g., water) for injection, immediately prior to use. Extemporaneous injection solutions and suspensions can be prepared from sterile powders, granules, or tablets of the kind previously described.

The compounds described herein in some embodiments can be formulated with additives selected from sodium acetate, acetic acid, ethanol, glycerinum, poloxamer, polyethylene glycol hydroxyl stearate, glycine, L-cysteine hydrochloride, hydroxypropyl beta cyclodextrins, and water. In some embodiments, the formulation comprises additives selected from sodium acetate, acetic acid, and water. For example, in some embodiments, the formulation comprises the compound of Formula I and sodium acetate. In some embodiments, the formulation comprises the compound of Formula I and an acetic acid. In some embodiments, the formulation comprises the compound of Formula I and water. In some embodiments, the formulation comprises the compound of Formula I, sodium acetate, and acetic acid. In some embodiments, the formulation comprises the compound of Formula I, sodium acetate, and water. In some embodiments, the formulation comprises the compound of Formula I, acetic acid, and water. In some embodiments, the formulation comprises the compound of Formula I, sodium acetate, acetic acid, and water

In some embodiments, the compound is present in a pharmaceutical composition comprising about any of 0.01M, 0.05M, 0.1M, 0.12M, 0.15M, or 0.2M sodium acetate/acetic buffer solution. In some embodiments, the composition comprises about 0.1M sodium acetate/acetic buffer solution.

### Articles of Manufacture and Kits

The present disclosure further provides kits for carrying out the methods of the invention, which comprises one or more compounds described herein or a composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for the treatment any disease or described herein, for example for the treatment of cancer.

Kits of the invention include one or more containers comprising the compound of Formula I, and in some embodiments, further comprise instructions for use in accordance with any of the methods described herein. The kit may further comprise a description of selection an individual suitable or treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of the invention are in suitable packaging. Suitable packaging include, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the compositions generally include information as to dosage, dosing schedule, and route of administration for the intended treatment. The containers may be unit doses, bulk packages (e.g., multi-dose packages) or sub-unit doses For example, kits may be provided that contain sufficient dosages of the compound of Formula I as disclosed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compound of Formula I and pharmaceutical compositions and instructions for use and packaged in quantities sufficient for storage and use in pharmacies, for example, hospital pharmacies and compounding pharmacies.

In some embodiments, the kit comprises the compound of Formula I and instructions for administering the compound of Formula I for the effective treatment of cancer.

The kits, medicines, and compositions of this invention may include any one or more aspects or parameters described herein.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Example 1. Phase I Study of an Exemplary Compound

A phase I study of an exemplary compound of Formula I (such as any one of the compounds listed in Table 1) or a pharmaceutically acceptable salt thereof is conducted with locally advanced or metastatic solid tumors.

The primary objectives of the study is to evaluate the safety and tolerability of an exemplary compound of Formula I administered 3 times weekly (Days 1, 2, and 3 for three weeks followed by one week of rest) by intravenous (IV) infusion to patients with locally advanced or metastatic solid tumors and to determine the Maximum Tolerated Dose (MTD) of an exemplary compound of Formula I. If an MTD is not reached, an optimum biological dose (OBD) is determined after the Safety Review Committee (SRC) and Investigators discuss the optimal balance toxicity, pharmacokinetics (PK), pharmacodynamics (PD), and clinical response signals.

The secondary objectives of the study is to characterize the PK of an exemplary compound of Formula I administered by repeat IV infusion in patients with locally advanced or metastatic solid tumors, to investigate potential biomarkers (e.g., PI3K and pAKT) for the action of an exemplary compound of Formula I, and to evaluate the preliminary efficacy of an exemplary compound of Formula I including objective response rate by Response Evaluation Criteria In Solid Tumors (RECIST) 1.1 criteria and duration of response.

The Phase I, open-label study is conducted on an exemplary compound of Formula I administered 3 times weekly for 3 weeks (Days 1, 2, and 3 of each week followed by one week of rest) via IV infusion to patients with locally advanced or metastatic solid tumors for whom standard therapy either does not exist or has proven to be ineffective or intolerable.

The first part of the study is on dose escalation, which uses a standard 3+3 design. The starting dose to be administered 3 times weekly is determined based on the PK and safety findings from earlier clinical studies. The starting dose is 0.4 mg/m² of an exemplary compound of Formula I per day and subsequent dose escalation proceeds with dose increases of 20-100% between cohorts. Doses may be increased by 100% in early cohorts; however, the increment for dose escalation is determined based on the safety data. Specifically, once a treatment-related Grade 2 or higher toxicity or dose-limiting toxicity (DLT) occurs, dose increments between cohorts are no greater than 50%. Dosing is continued until the MTD is reached to provide a recommended Phase II dose (RP2D). Intermediate dose levels and alternative dosing schedules may be investigated based on the emerging safety, PK, and efficacy data from earlier dosing cohorts. Patients are monitored for safety in the treatment unit on Days 1, 2 and 3 of Cycle 1 until 6 hours after the start of the infusion by the study staff. For subsequent infusions, patient discharge is based on clinical observations—patients can be discharged home at the end of the infusion if no electrocardiogram (ECG) changes or other signs or symptoms are observed. Each treatment cycle is 28 days, composed of 3 doses per week (Days 1, 2, and 3 each week) for three weeks and then one week of rest. Dose escalation and the opening of the next cohort occur only after acceptable tolerance has been demonstrated throughout the entire Cycle 1 and the DLT window period of 28 days. The decision is approved by the SRC.

The second part of the study is on dose expansion. The expansion part consists of all solid tumors and up to an additional three (3) cohorts based on specific tumor type(s) of 20 patients each treated at the RP2D. The goal of this cohort expansion is to confirm the toxicity and PK profile at the RP2D dose in each of the tumor types selected and to obtain expanded biomarker data. The final sample size may vary depending on the number of dose levels evaluated and the number of DLT's observed in each cohort. All patients are carefully followed for adverse events (AEs) during the study treatment and for 30 days after the last dose of study drug. AEs is graded according to the National Cancer Institute Common Terminology Criteria for Adverse Events (NCI CTCAE), Version 4.03. Dosing with an exemplary compound of Formula I is continued as long as there is no disease progression, prohibitive toxicity, or patient withdrawal. A study completion visit is performed within 4 weeks after the last dose of an exemplary compound of Formula I for all patients.

The investigational product of this study is an exemplary compound of Formula I. Injection of an exemplary compound of Formula I is provided as 5 mL vials (0.1 mg/mL) and is diluted in 0.9% sodium chloride to the desired concentration and is administered as a slow constant-rate 2-hour (±10 minutes) intravenous infusion.

Eligible patients for the study must meet the following criteria 1. sign Informed Consent Form; 2. be >18 years old: 3. exhibit histologically or cytologically documented cancer; 4. exhibit locally advanced, or metastatic solid malignancy that has progressed on, or failed at least one prior systemic therapy or have refused systemic treatment; 5. exhibit Eastern Cooperative Oncology Group (ECOG) performance status of 0 or 1; and 6. male or female patients of child-producing potential must agree to use double barrier contraception: condoms, sponge, foams, jellies, diaphragm or intrauterine device (IUD), contraceptives (oral, implants or injectable) or other avoidance of pregnancy measures during the study and for 90 days after the last day of treatment.

The average study duration for each patient is estimated to be up to approximately 7 months. The duration of the entire study is approximately 12-24 months.

Safety parameters for the study include physical examination, ECOG performance status, vital signs, 12-lead electrocardiograms (ECGs), AEs, and clinical laboratory testing.

Tumor response is determined through imaging (computed tomography (CT) or magnetic resonance imaging (MRI)) at baseline for all patients and every 2 cycles for the first 6 cycles, then every 3 cycles thereafter. Response will be assessed using Response Evaluation Criteria In Solid Tumors (RECIST—Version 1.1). Other methods of response assessment are also used as applicable, including measurement of serum tumor markers as applicable. Tumor tissue will be analysed for the presence of biomarkers (e.g., PI3K and pAKT).

A full PK profile is collected in Cycle 1 for the Day 1 and Day 3 dose, and for the first cycle of any dose reduction. A sparse PK profile is collected in Cycles 1 to 3.

### Example 2. Continuous Dosing of an Exemplary Compound

In this proposed study, a similar clinical trial is conducted with an exemplary compound of Formula I as discussed in Example 1, except that the compound is administered continuously for 24 hours on days 1-14 followed by one week of rest. The safety and efficacy of the compound is evaluated as discussed in Example 1.

### Example 3. Clinical Trial of an Exemplary Compound

This example provides results from a clinical trial which studied the efficacy of an exemplary compound of Formula I or a pharmaceutically acceptable salt thereof on various tumor types. An exemplary compound of Formula I was administered once a week for three weeks via a 2 hour intravenous (IV) infusion to patients. A treatment cycle was defined as 28 days, composed of 21 days of treatment and 7 days of rest. The number of completed treatment cycles ranged from one to four. The dose of the exemplary compound of Formula I ranged from 0.08 to 1.0 mg/m². Patients for the study included both males and females. The tumor type investigated in this clinical study included colorectal, breast, liver, gastric, and non-small cell lung cancer. Efficacy assessment was determined at the last patient visit of the study. The results are shown in Table 2 below.

TABLE 2

| Exemplary | No. of | | | |
|---|---|---|---|---|
| compound of | Completed | | | Efficacy |
| Formula 1 Dose | Treatment | | Tumor | Assessment |
| (mg/m²) | Cycles | Gender | Type | at Last Visit |
| 0.08 | 3 | Male | Colorectal | SD |
| 0.13 | 2 | Female | Colorectal | SD |
| 0.27 | 3 | Male | Colorectal | PD (SD at end |
| | | | | of Cycle 2) |
| 0.36 | 2 | Female | Breast | PD |
| 0.54 | 4 | Male | Colorectal | PD (SD at end |
| | | | | of Cycle 2) |
| 0.81 | 4 | Male | Liver | PD (SD at end |
| | | | | of Cycle 2) |
| 1.0 | 1 | Female | Gastric | SD |
| 1.0 | 1 | Female | Non-Small | SD |
| | | | Cell Lung | |

Abbreviations in Table 2: SD, stable disease; and PD, progressive disease.

In the analysis of the available safety data, the exemplary compound of Formula I was generally well tolerated when administered once a week by slow constant-rate IV infusion.

1. A method of treating cancer in an individual, comprising administering to the individual an effective amount of a compound of Formula I: 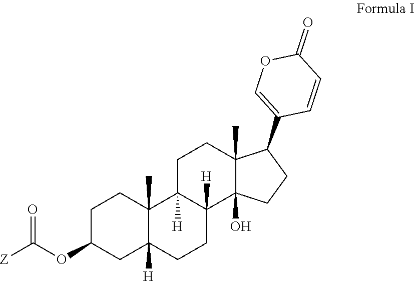
or a pharmaceutically acceptable salt thereof, wherein:
Z is —OR¹ or —NR²R³;
R¹ is optionally substituted alkyl, optionally substituted cycloalkyl, optionally substituted heterocycloalkyl, optionally substituted aryl, or optionally substituted heteroaryl; R² is hydrogen, optionally substituted alkyl, optionally substituted cycloalkyl, optionally substituted heterocycloalkyl, optionally substituted aryl, or optionally substituted heteroaryl; and R³ is optionally substituted alkyl, optionally substituted cycloalkyl, optionally substituted heterocycloalkyl, optionally substituted aryl, or optionally substituted heteroaryl, or R² and R³ are taken together with the atom to which they are attached to form an optionally substituted heterocycloalkyl;
and the compound is administered at least once a week for at least two weeks.

2. The method of claim 1, wherein the compound is administered at least two times a week.

3. The method of claim 1, wherein the compound is administered at least three times a week.

4. The method of claim 3, wherein the compound is administered for at least three consecutive days during a week.

5. The method of claim 3, wherein the compound is administered once every other day during a week.

6. The method of claim 3, wherein the compound is administered three times a week.

7. The method of claim 4, wherein the compound is administered on days 1, 2, and 3 of each week.

8. The method of claim 5, wherein the compound is administered on days 1, 3, and 5 of each week.

9. The method of claim 3, wherein the compound is administered daily for at least two weeks.

10. The method of claim 1, wherein the compound is administered at least once a week for at least two weeks.

11. The method of claim 1, wherein the compound is administered at least once a week for at least three weeks.

12. The method of claim 10, wherein the compound is administered at least once a week for two weeks with one week of rest in a 21-day dosage cycle.

13. The method of claim 11, wherein the compound is administered at least once a week for three weeks with one week of rest in a 28-day dosage cycle.

14. (canceled)

15. The method of claim 1, wherein the compound is administered at the dosage range of about 0.2 mg/m² to about 2 mg/m².

16. The method of claim 1, wherein the total dose of the compound administered to the individual is at least about 0.4 mg/m² per week.

17. The method of claim 1, wherein the cancer is selected from the group consisting of colorectal cancer, liver cancer, lung cancer, breast cancer, prostate cancer, pancreatic cancer, and oral cancer.

18. The method of claim 1, wherein the cancer is selected from the group consisting of colorectal cancer, breast cancer, liver cancer, gastric cancer, and non-small cell lung cancer.

19. The method of claim 1, wherein the cancer is locally advanced or metastatic cancer.

20. The method of claim 1, wherein the compound is administered intravenously.

21-35. (canceled)

36. The method of claim 1, wherein the compound is selected from Compound Nos. 1-7, or a pharmaceutically acceptable salt thereof: 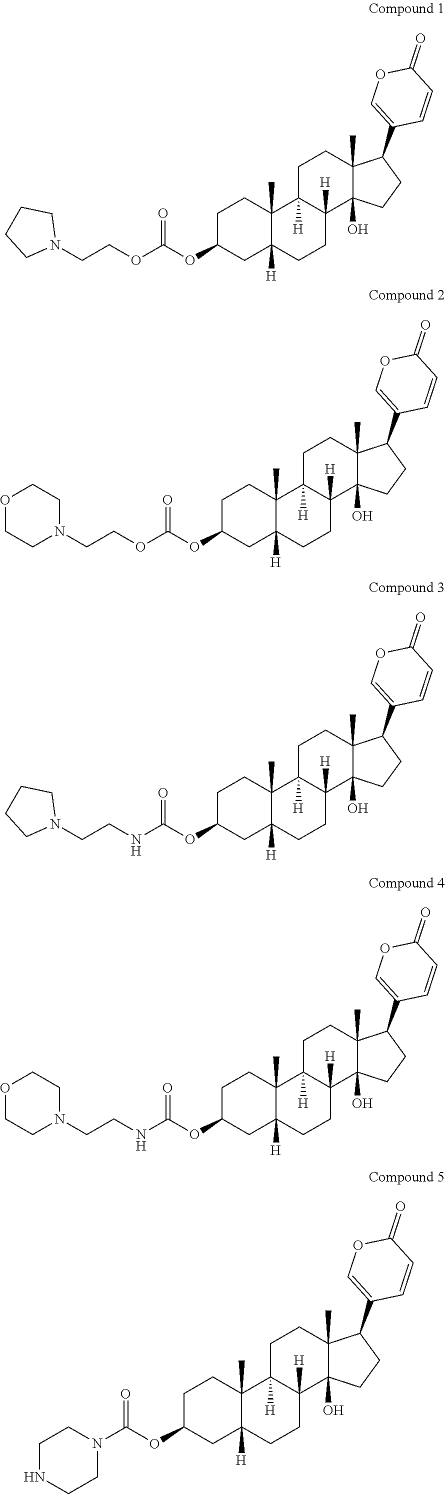 [chemical structure]
